(19) United States
(12) Patent Application Publication
(10) Pub. No.: US 2014/0193867 A1
(43) Pub. Date: Jul. 10, 2014

(54) MICROBIAL ODD CHAIN FATTY ACIDS

(71) Applicant: William Marsh Rice University, Houston, TX (US)

(72) Inventors: Ka-Yiu SAN, Houston, TX (US); Hui WU, Houston, TX (US)

(21) Appl. No.: 14/104,628

(22) Filed: Dec. 12, 2013

Related U.S. Application Data
(60) Provisional application No. 61/740,959, filed on Dec. 21, 2012.

(51) Int. Cl. C12P 7/64

(52) U.S. Cl. CPC C12P 7/6409
USPC 435/134; 435/252.3; 435/252.33

(57) ABSTRACT

Engineered bacteria that produce a predominance of odd-chain free fatty acids.

### PRIOR RELATED APPLICATIONS

This application claims priority to 61/740,959, filed Dec. 21, 2012, and incorporated by reference herein in its entirety.

### FEDERALLY SPONSORED RESEARCH STATEMENT

This invention was made with government support under Grant No: EEC-0813570 awarded by the NSF. The government has certain rights in the invention.

### FIELD OF THE DISCLOSURE

The present disclosure relates to microbial production of odd-chain free fatty acids.

### BACKGROUND OF THE DISCLOSURE

Biodiesel is considered to be an alternative to petroleum-derived diesel fuel, consisting of fatty acid methyl esters (FAMEs) and fatty acid ethyl esters (FAEEs). The changing fatty acid composition will be a useful tool for further modification of the biodiesel quality.

In addition, odd-chain fatty acids have been used in cosmetics and flagrance. For example, pelargonic acid (C9 fatty acid and its derivatives) for use in food, cosmetics, shampoos and other personal care products, and in transdermal drug delivery systems. Other applications are as a developer for photographic plates, in lacquers and plastics, and in lubricants and metalworking fluids.

Bacteria synthesis of fatty acids generally occurs with the following 6 reactions:

| Step | Enzyme | Reaction | Description |
|---|---|---|---|
| (a) | Acetyl CoA:ACP transacylase | 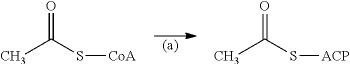 | Activates acetyl CoA for reaction with malonyl-ACP |
| (b) | Malonyl CoA:ACP transacylase | 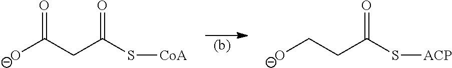 | Activates malonyl CoA for reaction with acetyl-ACP |
| (c) | 3-ketoacyl- ACP synthetase | 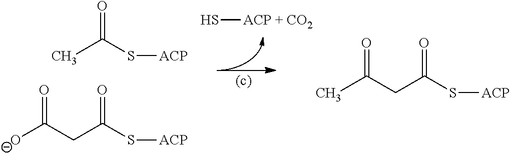 | Reacts priming acetyl- ACP with chain- extending malonyl- ACP. |
| (d) | 3-ketoacyl- ACP reductase | 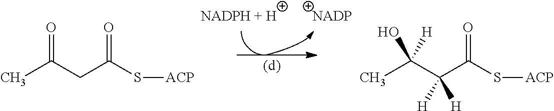 | Reduces the carbon 3 ketone to a hydroxyl group |
| (e) | 3- Hydroxyacyl ACP dehydrase | 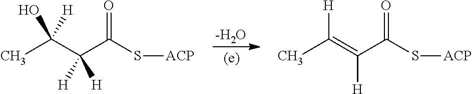 | Removes water |
| (f) | Enoyl-ACP reductase | 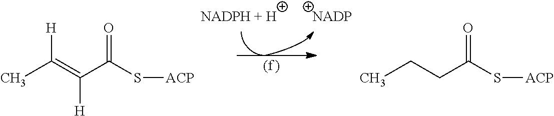 | Reduces the C3-C4 double bond. |

It is known that the addition of propionate to E. coli culture medium can allow the synthesis of odd-chain fatty acids, by introducing propionyl-CoA into the above pathway. However, under normal circumstances very little odd-chain fats are formed.

What is needed in the art are methods of making substantial amounts of odd-chain fats. Preferably a bacterial method of making predominantly odd-chain fats will be developed, thus providing a clean and sustainable method of producing these valuable chemicals.

### SUMMARY

The existing genetically engineered E. coli strains (see e.g., WO2011116279) produce higher levels of even numbered fatty acids than odd numbered fatty acids. We therefore further modified bacteria to make more odd-chain than even-chain fats. This was accomplished by manipulating the starting material to be a C3 molecule, propionyl-CoA, by overexpressing the propionyl-CoA synthase gene. We also replaced the native β-ketoacyl-acyl carrier protein synthase III gene with one having a greater substrate preference for propionyl-coA than acetyl-coA.

With these modifications, greater odd-chain fats were produced than was heretofore possible. In fact, >80% of the fats produced by such strains were of odd-chain lengths.

To manipulate the starting material, propionate can be provided in the nutrient medium. Alternatively, the bacteria can be made to provide its own propionate, by incorporating genes that encode enzymes needed for propionate's synthesis.

The advantages of using the manipulation of the first step in fatty acid elongation cycle to produce odd-chain free fatty acids include:
Increased intracellular level of propionyl-CoA.
Produced short, medium and long chain of odd-chain free fatty acids
Increased ratio of odd-chain free fatty acids to even-chain free fatty acids.
Use of mixed culture to combine the propionate production process with the fatty acid production process

The steps involved in creating the modified strains can easily be repeated in other strains or using genes from other species. The steps were basically:
Amplification and cloning of a propionyl-CoA synthase gene.
Confirmation of the DNA sequence of the cloned propionyl-CoA synthase gene.
Amplification and cloning of β-ketoacyl-acyl carrier protein synthase III genes with higher specificity for propionate, e.g., from Bacillus subtilis (fabH1), Bacillus subtilis (fabH2), Staphylococcus aureus (fabH) and Streptomyces peucetius (dpsC). Additional enzymes can be selected based on homology, and/or screened for substrate preference. Preferably, this gene is added to the plasmid which containing the propionyl-CoA synthase gene.
Confirmation of the DNA sequence of the cloned β-ketoacyl-acyl carrier protein synthase III gene.
Knockout of the expression of native β-ketoacyl-acyl carrier protein synthase III gene. This step is optional, but will usually improve yields by reducing the competition.
Transform the expression vector into the appropriate strain.
Optimize the induction of these enzymes and the initial concentration of propionate.
Confirm cell growth, substrate consumption, and fatty acid production.

A number of variations or modifications can be made to produce odd-chain fatty acid.
1. Overexpression of the native propionyl-CoA synthase
2. Identify heterologous or mutant propionyl-CoA synthase with increased activity
3. Reduction of EMP pathway (Embden-Meyerhof-Parnas glycolytic pathway) to reduce the competition of acetyl-CoA with propionyl-CoA
4. Knockdown the expression of native β-ketoacyl-acyl carrier protein synthase III gene
5. Identify heterologous or mutant β-ketoacyl-acyl carrier protein synthase III with increased specificity of propionyl-CoA and activity
6. Options of propionate supply method: (1) Adding propionate directly in the medium; (2) Introduce a heterologous propionate synthesis pathway or overexpress a native one; (3) Combine methods 1 and 2.

In various embodiments, the invention comprises one or more of the following embodiments, in any combination thereof:

A genetically modified bacteria, said bacteria having:
an overexpressed propionyl-CoA synthase gene;
an overexpressed β-ketoacyl-acyl carrier protein synthase III gene with a greater substrate preference for propionyl-coA than acetyl-coA; and
optional knockout of a native β-ketoacyl-acyl carrier protein synthase III gene.
Note: Taking this gene out may not be essential, depending on the comparative activities and desired fatty acid profile. Hence, its removal is considered optional, and it can be reduced or left as is if the competition level is acceptable.

A genetically modified bacteria, said bacteria having:
a) an overexpressed propionyl-CoA synthase gene, e.g., from Salmonella enterica;
b) an overexpressed β-ketoacyl-acyl carrier protein synthase III gene from Bacillus subtilis (fabH1), Bacillus subtilis (fabH2), Staphylococcus aureus (fabH) or Streptomyces peucetius (dpsC); and
c) knockout of a native β-ketoacyl-acyl carrier protein synthase III gene.

A bacteria wherein overexpressed genes are co-expressed from the same expression vector.

A bacteria as described herein further comprising a knockout of the native fadD.

A bacteria as described herein further comprising an overexpressed acyl-ACP thioesterase, e.g., from U. califonica, R. communis, or C. hookeriana or as described in WO2011116279.

A bacteria as described herein, further comprising Δack, Δpta or Δack-pta.

A genetically modified bacteria selected from Table 1-18 (excepting parental strains).

A genetically modified bacteria from Table 1-18 and producing more odd-chain fatty acids than comparable bacteria not so genetically modified.

A genetically modified bacteria producing at least 50, 60, 70, 80 or 90% odd-chain fatty acids.

A method of producing odd-chain fatty acids, comprising culturing any bacteria herein described in a nutrient broth for a period of time sufficient for said bacteria to produce odd-chain fatty acids, and isolating said odd-chain fatty acids from said nutrient broth, or said bacteria, or both.

Methods of making odd-chain fats by incubating the bacteria described herein and isolating odd-chain fats.

Methods wherein propionate is provided in said nutrient broth or wherein propionate is provided by overexpressing the genes from Propionibacterium encoding for oxaloacetate transcarboxylase and succinyl CoA transferase, thus allowing the production of propionic acid from pyruvate, or combinations thereof.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

As used herein, “fatty acids” means any saturated or unsaturated aliphatic acids having the common formulae of CₙH_{2n±x}COOH, wherein x≦n, which contains a single carboxyl group. “Odd-chain” fatty acids have an odd number of carbons in the chain (n=even).

As used herein, “reduced activity” is defined herein to be at least a 75% reduction in protein activity, as compared with an appropriate control species. Preferably, at least 80, 85, 90, 95% reduction in activity is attained, and in the most preferred embodiment, the activity is eliminated (100%). Proteins can be inactivated with inhibitors, by mutation, or by suppression of expression or translation, by knock-out, by adding stop codons, by frame shift mutation, and the like.

By “knockout” or “null” mutant, what is meant is that the mutation produces almost undetectable amounts of protein activity. A gene can be completely (100%) reduced by knockout or removal of part of all of the gene sequence. Use of a frame shift mutation, early stop codon, point mutations of critical residues, or deletions or insertions, and the like, can also completely inactivate (100%) gene product by completely preventing transcription and/or translation of active protein. All knockout mutants herein are signified by Δgene.

As used herein, “overexpression” or “overexpressed” is defined herein to be at least 150% of protein activity as compared with an appropriate control species. Preferably, the activity is increased 200-500%. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like. All overexpressed genes or proteins are signified herein by “+”.

As used herein, all accession numbers are to GenBank unless indicated otherwise.

Exemplary gene or protein species are provided herein. However, gene and enzyme nomenclature varies widely, thus any protein (or gene encoding same) that catalyzes the same reaction can be substituted for a named protein herein. Further, while exemplary protein sequence accession numbers are provided herein, each is linked to the corresponding DNA sequence, and to related sequences. Further, related sequences can be identified easily by homology search and requisite activities confirmed as by enzyme assay, as is shown in the art.

E. coli gene and protein names (where they have been assigned) can be ascertained through ecoliwiki.net/ and enzymes can be searched through brenda-enzymes.info/. ecoliwiki.net/ in particular provides a list of alternate nomenclature for each enzyme/gene. Many similar databases are available including UNIPROTKB, PROSITE; 5 EC2PDB; ExplorEnz; PRIAM; KEGG Ligand; IUBMB Enzyme Nomenclature; IntEnz; MEDLINE; and MetaCyc, to name a few.

By convention, genes are written in italic, and corresponding proteins in regular font. E.g., fadD is the gene encoding FadD or acyl-CoA synthetase.

Generally speaking, we have used the gene name and protein names interchangeably herein, based on the protein name as provided in ecoliwiki.net. The use of a protein name as an overexpressed protein (e.g., FabH+) signifies that protein expression can occur in ways other than by adding a vector encoding same, since the protein can be upregulated in other ways. The use of FadD signifies that the protein can be downregulated in similar way, whereas the use of ΔfadD means that the gene has been directly downregulated, e.g., by knockout or null mutation.

Note: The fadD mutation used herein was for convenience only. It is incidental to the invention.

### DESCRIPTION OF FIGURES

FIG. 1. Diagram of engineered pathways.

FIG. 2. Schematic diagram of the prpE overexpression plasmid pBAD33-prpE. The 1887 bp of gene encoded propionyl-CoA synthase (prpE) from Salmonella enterica was amplified by PCR and cloned into the vector pBAD33. Abbreviations: ara promoter, arabinose induced promoter; araC, regulator gene of arabinoase promoter; CmR, chloramphenicol resistant gene; pACYC184 ori, origin of replication of plasmid pACYC184; rrnBT1,2, transcriptional terminator of rrnB.

FIG. 3A-D. Schematic diagrams of the fabHs and prpE co-overexpression plasmids, pBHE1 (A), pBHE2 (B), pBHE3 (C) and pBHE4 (D). (A) The 978 bp of gene encoded the β-Ketoacyl-Acyl Carrier Protein Synthase B from Bacillus Subtilis (bs_fabH2) was synthesized and cloned into the vector pBAD33-prpE. (B) The 967 bp of gene encoded the β-Ketoacyl-Acyl Carrier Protein Synthase from Staphylococcus aureus (sa_fabH) was synthesized and cloned into the vector pBAD33-prpE. (C) The 936 bp of gene encoded the β-Ketoacyl-Acyl Carrier Protein Synthase A from Bacillus Subtilis (bs_fabH1) was synthesized and cloned into the vector pBAD33-prpE. (D) The 1062 bp of gene encoded the β-Ketoacyl-Acyl Carrier Protein Synthase subunit from Streptomyces peucetius (sp_dpsC) was synthesized and cloned into the vector pBAD33-prpE. Abbreviations: Prom a, trc promoter; araC; CmR, chloramphenicol resistant gene; pACYC 184 ori, origin of replication of plasmid pACYC 184; rrnBT 1,2, transcriptional terminator of rrnB.

FIG. 4A-D. Schematic diagrams of the fabH overexpression plasmids, pBH1 (A), pBH2 (B), pBH3 (C) and pBH4 (D). (A) The 978 bp of gene encoded the β-Ketoacyl-Acyl Carrier Protein Synthase B from Bacillus Subtilis (bs_fabH2) was amplified by PCR using pBHE1 as the template and cloned into the vector pBAD33. (B) The 967 bp of gene encoded the β-Ketoacyl-Acyl Carrier Protein Synthase from Staphylococcus aureus (sa_fabH) was amplified by PCR using pBHE2 as the template and cloned into the vector pBAD33. (C) The 936 bp of gene encoded the β-Ketoacyl-Acyl Carrier Protein Synthase A from Bacillus Subtilis (bs_fabH1) was amplified by PCR amplified by PCR using pBHE3 as the template and cloned into the vector pBAD33. (D) The 1062 bp of gene encoded the β-Ketoacyl-Acyl Carrier Protein Synthase subunit from Streptomyces peucetius (sp_dpsC) was amplified by PCR using pBHE4 as the template and cloned into the vector pBAD33. Abbreviations: Prom a, trc promoter; araC; CmR, chloramphenicol resistant gene; pACYC184 ori, origin of replication of plasmid pACYC184; rrnBT1,2, transcriptional terminator of rrnB.

FIG. 5 showing the conversion of pyruvate to propionate via four pathways in the propioni bacteria.

### DETAILED DESCRIPTION

The present disclosure establishes an in vivo method for the microbial production of odd-chain free fatty acid that increases the concentration, increases the yield, and increases the percentage.

To demonstrate, we use the previously engineered high even-chain fatty acid producing E. coli strain ML103 (pXZ18), ML103 (pXZM12) and ML191 (pXZmch). The ML103 (pXZ18) possessed acyl-CoA synthetase (fadD) mutation and overexpression of acyl-ACP thioesterase from Ricinus communis; the overexpressed acyl-ACP thioesterase in the ML103 (pXZM12) came from Umbellularia califonica. The ML191 (pXZmch) had inactivated acyl-CoA synthetase (fadD) and phosphofructokinase (pfkA) and overexpressed the acyl-ACP thioesterase from Cuphea hookeriana. See WO2011116279 for a variety of additional TE protein sequences.

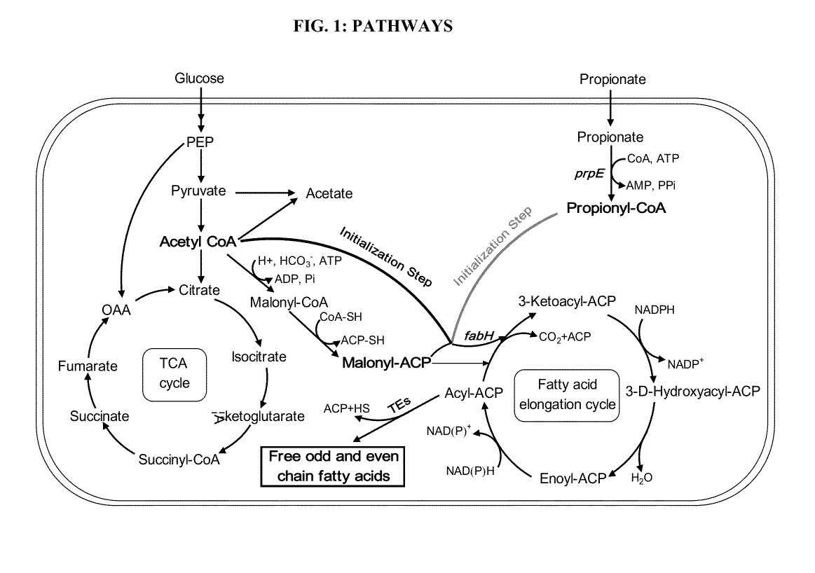

These strains were enhanced with the heterologous expression of propionyl-CoA synthase (prpE) from Salmonella enterica to improve intracellular propionyl-CoA availability. Additionally, co-expression of β-ketoacyl-acyl carrier protein synthase III (KAS III, fabH) from different sources, such as Bacillus subtilis (bs_fabH1), Bacillus subtilis (bs_fabH2), Staphylococcus aureus (Sa_fabH) and Streptomyces peucetius (sp_dpsC), together with knockout of native fabH to further manipulate the first step in the fatty acid elongation cycle (FIG. 1).

The accession numbers for the sequences actually used herein are as follows:

| Strain | Gene | Gene ID | Protein_ID |
|---|---|---|---|
| Salmonella enterica | prpE | 1251890 | AFD57404.1 |
| Bacillus subtilis | fabH1^{a} | 936392 | CAB12974.1 |
| Bacillus subtilis | fabH2^{b} | 939306 | CAB12857.1 |
| Staphylococcus aureus | fabH | 1120958 | BAB57145.1 |
| Streptomyces peucetius | dpsC | L35560.1 | AAA65208.1 |
| Escherichia coli | fabH | 946003 | AAC74175.1 |
| E. coli | pfkA | 948412 | AAC76898.1 |
| E. coli | fadD | 946327 | AAC74875.1 |

^{a}The name of the protein encoded by fabH1 is FabHA
^{b}The name of the protein encoded by fabH2 is FabHB

The strategy described herein has yet not been performed in other species, such as yeast, cyanobacteria, and microalgae. However, we predict that it can be applied to such species because the cloning methodology is well known and understood and can be applied in any bacteria or yeast. Further, fatty acid synthesis have only the two types of pathways. Type II enzymes of the fatty acid synthesis cycle are constituted by four enzymes, which are shown in the previous table. In different species, the function of these enzymes are very similar, while the sequence maybe different. Hence, we predict the same strategies should work with organism using type II fatty acid synthesis pathways.

Other exemplary sequences for propionyl-CoA synthase or propionyl-CoA ligase that could be used are as follows:

| | | % identity to |
|---|---|---|
| Strain | Protein_ID | AFD57404.1 |
| S. enterica | AFD57404.1 | 100% |
| Citrobacter rodentium | YP_003364035.1 | 92% |
| Citrobacter sp. KTE32 | WP_016151949.1 | 92% |
| Shigella flexneri | WP_005127937.1 | 91% |
| E. coli | WP_001652807.1 | 90% |
| Enterobacteriaceae | WP_000010296.1 | 82% |

Other exemplary β-ketoacyl-acyl carrier protein synthase III genes with a greater substrate preference for propionyl-coA than acetyl-coA include:

| | | % identity to |
|---|---|---|
| Strain | Protein_ID | BAB57145.1 |
| Staphylococcus aureus | BAB57145.1 | 100% |
| Bacillus subtilis | CAB12857.1 | 43% (59% positives) |
| Streptomyces peucetius | AAA65208.1 | 18% (39% positives) |
| Staphylococcus | WP_002508111 | 79% |
| Bacillus | WP_017551890 | 65% |
| Geobacillus | WP_008878983.1 | 62% |

Other potentially useful KAS III proteins that can be screened for their substrate preference include:

| | | % identity to |
|---|---|---|
| Strain | Protein_ID | CAB12857.1 |
| Bacillus subtilis | CAB12857.1 | 100% |
| B. amyloliquefaciens | YP_007446301.1\| | 81% |
| B. atrophaeus | YP_003972429.1 | 83% |
| Bacillus sonorensis | WP_006636704.1 | 73% |

| | | % identity to |
|---|---|---|
| Strain | Protein_ID | AAA65208.1 |
| Nocardia brasiliensis | YP_006806420.1 | 67% |

Endogenous β-ketoacyl-acyl carrier protein synthase III genes (fabH or KAS IIIs) that can be knocked out to reduce competition with the overexpressed propionate-preferring KAS III in the various host species are as follows:

| | | % identity to |
|---|---|---|
| Strain | Protein_ID | BAB57145.1 |
| E. coli | NP_287225.1 | 100% |
| Salmonella enterica | WP_023201810.1 | 95% |
| Citrobacter koseri | YP_001453530.1\| | 96% |
| Enterobacter cloacae | YP_006477147.1\| | 92% |

As can be seen, fatty acid synthesis related proteins are well conserved across species, and, therefore, it is very likely that the invention can be produced in other species, such as the Bacillus, Staphylococcus, Streptomyces, Salmonella, Cyanobacteria, microalgae, yeast and the like. Further, since the entire genomes of thousands of bacteria have been sequenced and annotated, it is simple to choose relevant sequence by either homology or EC number or name, and thus modify other bacteria as described herein.

The production of odd-chain free fatty acid in genetically engineered Escherichia coli strain is achieved via the expression of propionyl-CoA synthase (prpE) to form propionyl-CoA from extracellular propionate combined with expression of exogenous β-ketoacyl-acyl carrier protein synthase III (KAS III, fabH) from different sources and knockout of native fabH to further manipulate the first step in the fatty acid elongation cycle.

In previous studies, it was demonstrated that propionate can induced the synthesis of odd-chain-length fatty acids by Escherichia coli but the amount produced was very low (Ingram et al., 1977).

The overexpression of propionyl-CoA synthase (prpE) from Salmonella enterica was applied in poly (3-hydroxybutyrate-co-3-hydroxyvalerate) (PHBV) production by improving the intracellular propionyl-CoA levels (Aldor and Keasling, 2001; Wong et al., 2008).

The native β-ketoacyl-acyl carrier protein synthase, which catalyzes the first step in fatty acid elongation cycle, prefers acetyl-CoA as its substrate. The β-ketoacyl-acyl carrier protein synthases from Bacillus subtilis (bs_fabH1, bs_fabH2), Staphylococcus aureus (Sa_fabH), Streptomyces peucetius (sp_dpsC) and others prefer other substrates, such as isobutatyryl-CoA, propionyl-CoA and other acyl-CoAs, more than acetyl-CoA.

Some of the FabHs were already used for branch chain fatty acid production (Choi, et al., 2000; Smirnova and Reynolds, 2001; Zhihao Hu, et al., 2012). However, they have not previously been used as described herein.

The parent strains (ML103 pXZ18, ML103 pXZM12 and 191 pXZmch) used in this demonstration are capable of producing short, medium and long chain fatty acids from glucose to average concentrations of 2-3 g/L with yields of 13.3%-20% (Zhang, et al., 2011; San et al., 2011). However, no significant odd-chain free fatty acids are produced by these parent strains.

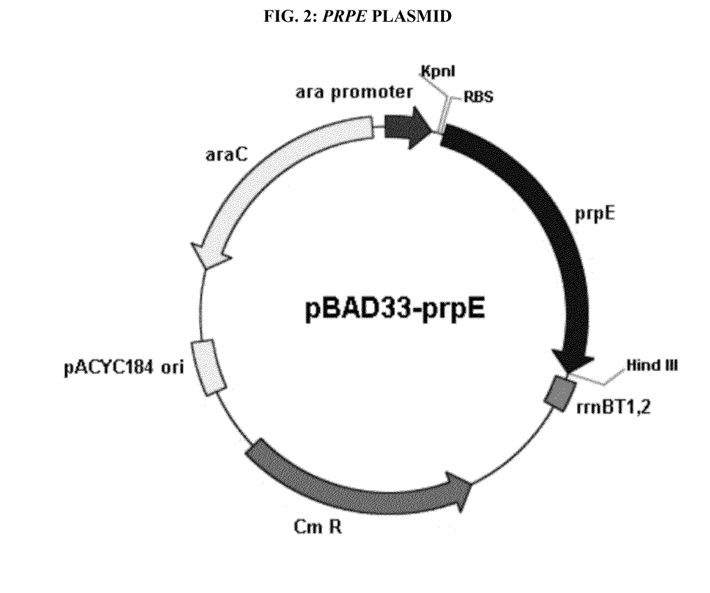

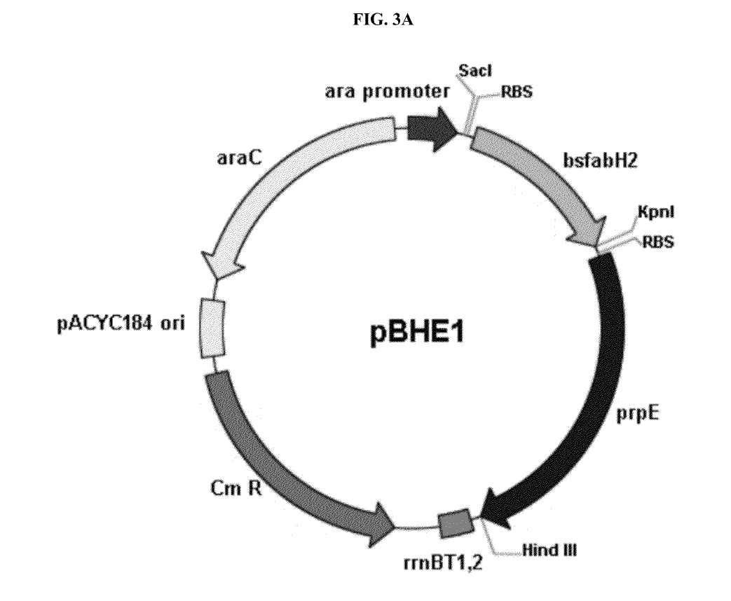

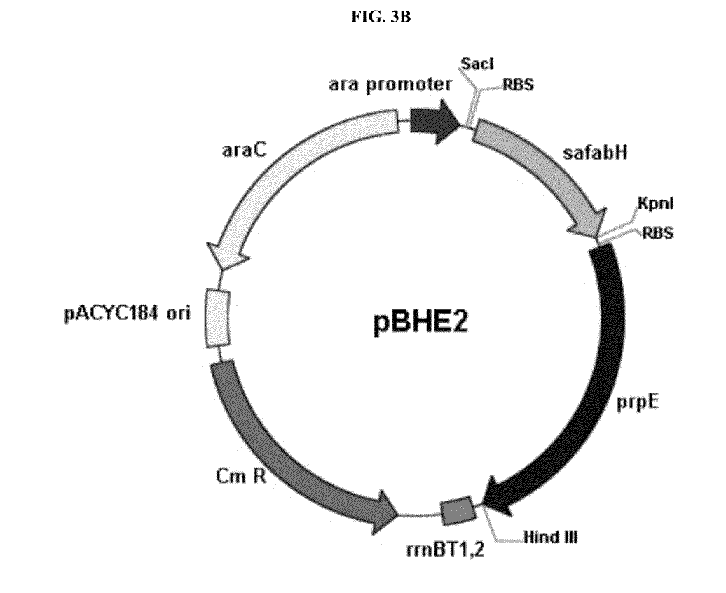

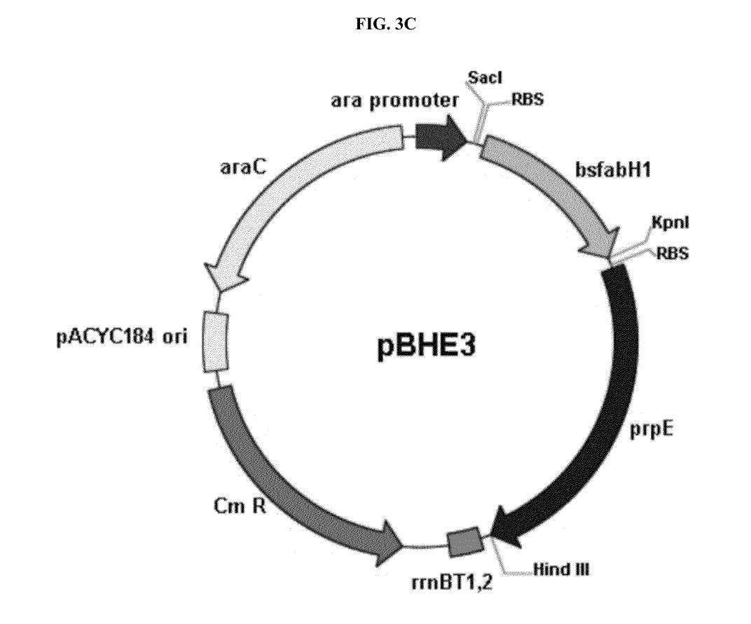

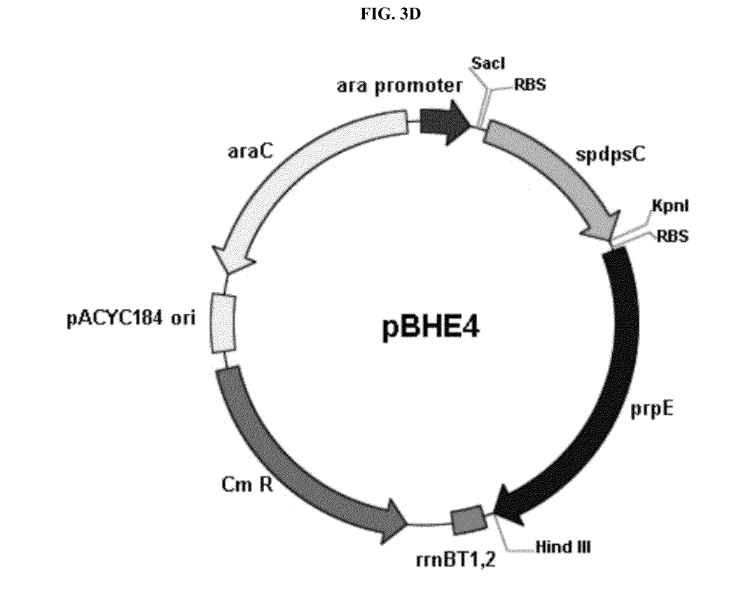

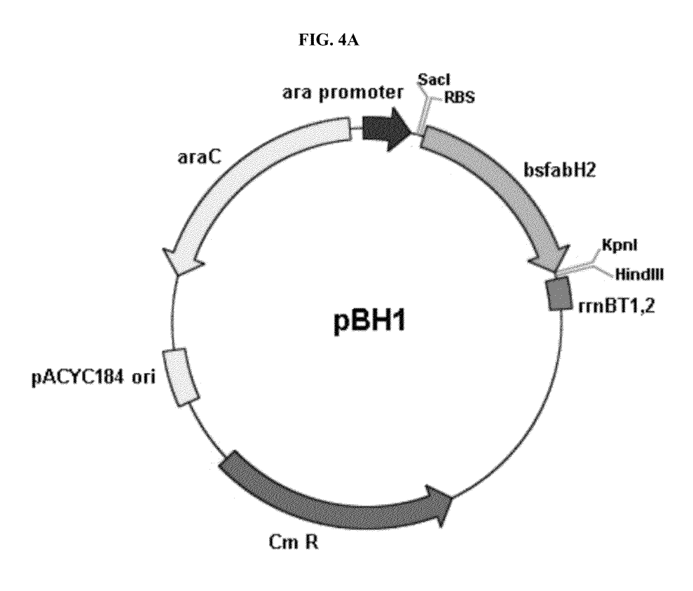

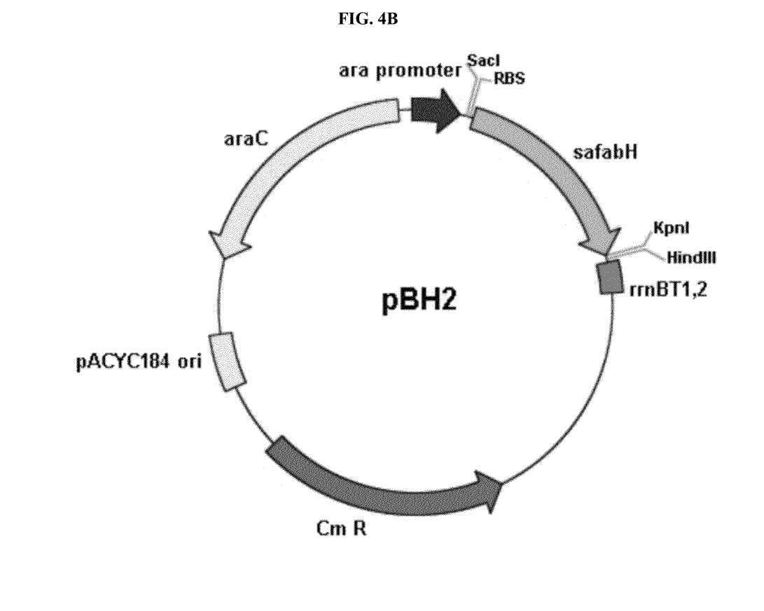

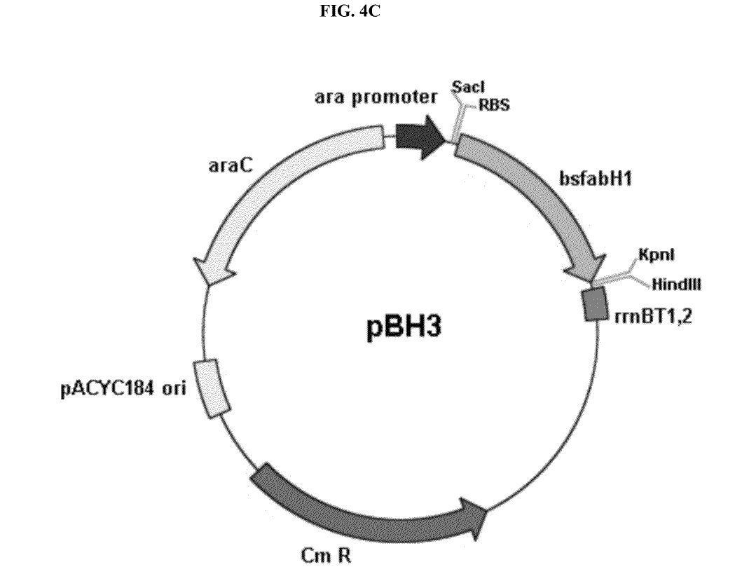

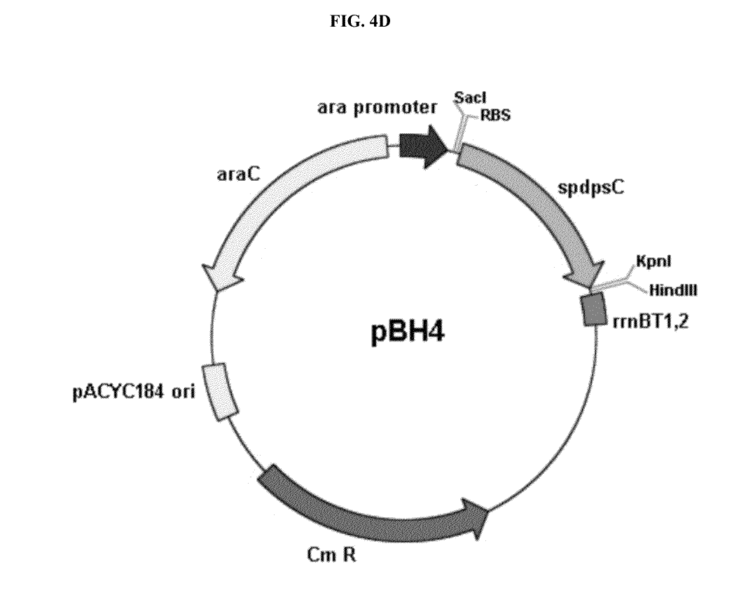

In order to accumulate the odd-chain free fatty acids, we constructed multiple vectors to overexpress propionyl-CoA synthase (prpE) from Salmonella enterica (FIG. 2), KAS Ms from different sources, such as Bacillus subtilis (bs_fabH1), Bacillus subtilis (bs_fabH2), Staphylococcus aureus (Sa_fabH) and Streptomyces peucetius (sp_dpsC) (FIG. 4) and co-overexpressed both prpE and fabHs (FIG. 3).

The newly introduced propionyl-CoA synthase is used to increase the intracellular propionyl-CoA availability when propionate is supplied in the medium, and the heterogenous KAS IIIs will enhance the substrate specificity of propionyl-CoA in the first step in the fatty acid elongation cycle.

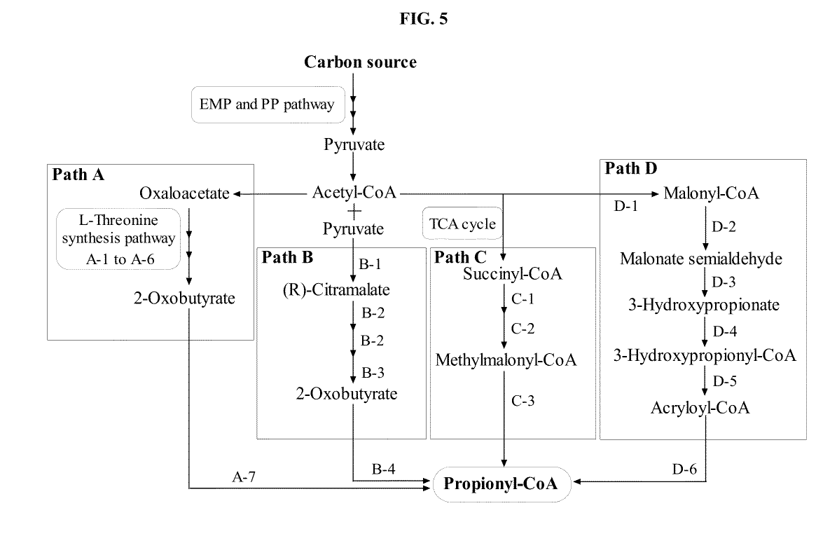

The native KAS III, which prefers acetyl-CoA, was knocked out to eliminate its competition effect with heterogenous KAS Ms. The functionality of odd-chain free fatty acid production systems were successfully demonstrated in shake flask fermentations (Tables 1-18).
It is also possible to add in the genes for propionate synthesis, which means that the bacteria can produce their own substrate f to make propionyl-coA. Bacteria normally produce propionyl-coA by four pathways, depicted in FIG. 5, which include threonine pathway (pathway A), citramalate pathway (pathway B), methylmalonyl-CoA pathway (pathway C), and 3-hydroxypropionate pathway (pathway D). The enzymes of these pathways are as follows:

| | Sequence | |
|---|---|---|
| Pathway | of reaction | Enzyme |
| A | A-1 | aspartate aminotransferase |
| | A-2 | aspartate kinase |
| | A-3 | aspartate semialdehyde dehydrogenase |
| | A-4 | homoserine kinase |
| | A-5 | threonine synthase |
| | A-6 | threonine deaminase |
| | A-7 | keto acid formate-lyase |
| B | B-1 | (R)-citramalate synthase |
| | B-2 | 3-isopropylmalate dehydratase |
| | B-3 | 3-isopropylmalate dehydrogenase |
| | B-4, A-7 | keto acid formate-lyase |
| C | C-1 | methylmalonyl-CoA mutase |
| | C-2 | methylmalonyl-CoA epimerase |
| | C-3 | methylmalonyl-CoA decarboxylase or |
| | | methylmalonyl-CoA carboxyltransferase or |
| | | biotin carboxylase |
| D | D-1 | acetyl-CoA carboxylase carboxyltransferase |
| | | or biotin carboxylase |
| | D-2 | malonyl-CoA reductase (NADPH) |
| | D-3 | malonate semialdehyde reductase (NADPH) |
| | D-4 | acyl-CoA synthetase |
| | | or 3-hydroxypropionate-CoA ligase |
| | D-5 | enoyl-CoA hydratase |
| | | or 3-hydroxypropionyl-CoA dehydratase |
| | D-6 | acryloyl-CoA reductase (NADPH) |

Suwannakham (2005) also optimized propionate production in Propionibacterium acidipropionici by reducing competition for carbons via acetate. The ack-deleted mutants (acc. no. AY936474) improved propionic acid production by ˜10% with a corresponding reduction in acetate formation. This suggests that Δack, Δpta or Δack-pta mutations would also be helpful.

### Experimental Methods

Aerobic shake flasks experiments were performed at 30° C. with shaking at 250 rpm for 24 hr or 48 hr with 10% inoculation in 50 ml LB broth medium supplied with 15 g/1 glucose and appropriate quantities of kanamycin, chloramphenicol and ampicillin.

The concentrations of IPTG of ML103 (pXZM12) and ML103 (pZX18) were 0.2 mM and 0.5 mM, respectively. Two IPTG concentrations were applied in the experiments of ML191ΔfabH (pXZmch, pBHE2). The initial pH was 7.5. Different concentrations of propionate and arabinose were investigated. Samples were taken at 24 hr and 48 hr.

The results are shown in Tables 1-17. The percentages of odd-chain free fatty acid were significantly improved in the new systems. As the control, dummy plasmids were transformed into the parent strains and it showed that the host strain produced negligible quantities of odd-chain fatty acid when different concentrations of propionate were supplied in the fermentation medium. The results of effect of prpE overexpression on odd chain fatty acid production are shown in Tables 1-6. The results of effect of different fabHs overexpression on odd chain fatty acid production are shown in Tables 7-12. The results of effect of prpE and different fabHs co-overexpression on odd chain fatty acid production are shown in Tables 13-18.

In particular, Tables 14 and 17 show several bacterial strains with odd-chain fatty acids of at least 50%, 60%, 70%, 80% and even higher than 90% odd-chain fatty acids.

TABLE 1
Concentration of free fatty acid production at 24 hr

| | | Culture Condition | | | | | | | | | | | | |
|---|---|---|---|---|---|---|---|---|---|---|---|---|---|---|
| | Relevant | Propionate | Arabinose | Concentrations of free fatty acid (g/L) | | | | | | | | | | |
| Strain | genotype | (mM) | (mM) | C10 | C11 | C12:1 | C12 | C13 | C14:1 | C14 | C15 | C16:1 | C16 | C18 |
| ML103 | ΔfadD, | 0 | 0 | 0.01 | 0 | 0.11 | 0.73 | 0 | 0.077 | 0.0379 | 0 | 0.011 | 0.014 | 0.026 |
| (pXZM12, | uc_TE⁺ | 8 | 0 | 0.0077 | 0.01 | 0.11 | 0.63 | 0.019 | 0.073 | 0.031 | 0.0018 | 0.0068 | 0.0092 | 0.019 |
| pBAD33) | | 12 | 0 | 0.0068 | 0.048 | 0.12 | 0.42 | 0.08 | 0.077 | 0.027 | 0.0044 | 0.012 | 0.0073 | 0.022 |
| ML103 | ΔfadD, | 8 | 5 | 0.007 | 0.035 | 0.12 | 0.59 | 0.067 | 0.081 | 0.033 | 0.0033 | 0.0061 | 0.0077 | 0.02 |
| (pXZM12, | uc_TE⁺, | 8 | 10 | 0.0074 | 0.046 | 0.12 | 0.57 | 0.086 | 0.077 | 0.032 | 0.0042 | 0.0072 | 0.0084 | 0.02 |
| pBAD33-prpE) | se_prpE⁺ | 8 | 20 | 0.0071 | 0.056 | 0.11 | 0.53 | 0.10 | 0.073 | 0.031 | 0.0058 | 0.0064 | 0.0092 | 0.02 |
| | | 12 | 10 | 0.0037 | 0.07 | 0.091 | 0.35 | 0.13 | 0.064 | 0.024 | 0.0072 | 0.0067 | 0.0079 | 0.015 |
| | | 12 | 20 | 0.0046 | 0.07 | 0.086 | 0.35 | 0.13 | 0.062 | 0.025 | 0.0069 | 0.0076 | 0.0077 | 0.018 |
| | | 12 | 50 | 0.0041 | 0.051 | 0.07 | 0.34 | 0.094 | 0.054 | 0.023 | 0.0049 | 0.0071 | 0.0075 | 0.018 |
| ML103 | ΔfadD, | 0 | 0 | 0 | 0 | 0 | 0 | 0 | 0 | 0.45 | 0 | 0.39 | 0.62 | 0.24 |
| (pXZ18, | rc_TE⁺ | 8 | 0 | 0 | 0 | 0 | 0 | 0 | 0 | 0.43 | 0.029 | 0.36 | 0.30 | 0.13 |
| pBAD33) | | 12 | 0 | 0 | 0 | 0 | 0 | 0.0023 | 0 | 0.46 | 0.059 | 0.43 | 0.38 | 0.220 |
| ML103 | ΔfadD, | 8 | 5 | 0 | 0 | 0 | 0 | 0.004 | 0 | 0.51 | 0.082 | 0.43 | 0.34 | 0.23 |
| (pXZ18, | rc_TE⁺, | 8 | 10 | 0 | 0 | 0 | 0 | 0.0025 | 0 | 0.48 | 0.076 | 0.41 | 0.36 | 0.19 |
| pBAD33-prpE) | se_prpE⁺ | 8 | 20 | 0 | 0 | 0 | 0 | 0.003 | 0 | 0.47 | 0.095 | 0.42 | 0.35 | 0.17 |
| | | 12 | 10 | 0 | 0 | 0 | 0 | 0.0045 | 0 | 0.42 | 0.11 | 0.39 | 0.28 | 0.12 |
| | | 12 | 20 | 0 | 0 | 0 | 0 | 0.006 | 0 | 0.37 | 0.13 | 0.32 | 0.23 | 0.096 |
| | | 12 | 50 | 0 | 0 | 0 | 0 | 0.0075 | 0 | 0.35 | 0.14 | 0.3 | 0.23 | 0.087 |

uc_TE⁺: overexpression of acyl-ACP thioesterase from Umbellularia californica;
rc_TE⁺: overexpression of acyl-ACP thioesterase from Ricinus communis;
se_prpE⁺: overexpression of propionyl-CoA synthetase from Salmonella enterica.

TABLE 2
Total fatty acid production and percentage of odd-chain fatty acid production at 24 hr

| | | | | Total odd- | Total even- | | Percentage of odd- |
|---|---|---|---|---|---|---|---|
| | Relevant | Culture Condition | | chain fatty | chain fatty | Total fatty | chain fatty acid |
| Strain | genotype | Propionate (mM) | Arabinose (mM) | acid (g/L) | acid (g/L) | acid (g/L) | (%) |
| ML103(pXZM12, pBAD33) | ΔfadD, uc_TE⁺ | 0 | 0 | 0 | 1.01 | 1.01 | 0 |
| | | 8 | 0 | 0.032 | 0.90 | 0.93 | 3.45 |
| | | 12 | 0 | 0.13 | 0.73 | 0.86 | 15.43 |
| ML103(pXZM12, pBAD33- | ΔfadD, uc_TE⁺, | 8 | 5 | 0.10 | 0.85 | 0.96 | 10.82 |
| prpE) | se_prpE⁺ | 8 | 10 | 0.14 | 0.83 | 0.97 | 14.00 |
| | | 8 | 20 | 0.17 | 0.78 | 0.95 | 17.45 |
| | | 12 | 10 | 0.21 | 0.56 | 0.77 | 26.94 |
| | | 12 | 20 | 0.21 | 0.56 | 0.77 | 26.83 |
| | | 12 | 50 | 0.15 | 0.52 | 0.67 | 22.38 |
| ML103(pXZ18, pBAD33) | ΔfadD, rc_TE⁺ | 0 | 0 | 0 | 1.69 | 1.69 | 0 |
| | | 8 | 0 | 0.029 | 1.22 | 1.25 | 2.34 |
| | | 12 | 0 | 0.062 | 1.5 | 1.56 | 3.94 |
| ML103(pXZ18, pBAD33- | ΔfadD, rc_TE⁺, | 8 | 5 | 0.086 | 1.52 | 1.6 | 5.36 |
| prpE) | se_prpE⁺ | 8 | 10 | 0.079 | 1.44 | 1.51 | 5.19 |
| | | 8 | 20 | 0.098 | 1.41 | 1.51 | 6.48 |
| | | 12 | 10 | 0.11 | 1.22 | 1.33 | 8.31 |
| | | 12 | 20 | 0.14 | 1.02 | 1.16 | 12.17 |
| | | 12 | 50 | 0.15 | 0.96 | 1.11 | 13.57 |

uc_TE⁺: overexpression of acyl-ACP thioesterase from Umbellularia californica;
rc_TE⁺: overexpression of acyl-ACP thioesterase from Ricinus communis;
se_prpE⁺: overexpression of propionyl-CoA synthetase from Salmonella enterica.

TABLE 3
Total glucose consumption, acetate accumulation and cell density at 24 hr

| | | Culture Condition | | | Acetate accumulation | |
|---|---|---|---|---|---|---|
| Strain | Relevant genotype | Propionate (mM) | Arabinose (mM) | Glucose consumption (mM) | (mM) | OD₆₀₀ |
| ML103(pXZM12, | ΔfadD, uc_TE⁺ | 0 | 0 | 52.08 | 8.15 | 5.23 |
| pBAD33) | | 8 | 0 | 52.91 | 4.41 | 4.84 |
| | | 12 | 0 | 56.08 | 1.63 | 5.12 |
| ML103(pXZM12, | ΔfadD, uc_TE⁺, | 8 | 5 | 54.66 | 6.63 | 5.38 |
| pBAD33-prpE) | se_prpE⁺ | 8 | 10 | 52.82 | 7.23 | 5.18 |
| | | 8 | 20 | 51.63 | 9.20 | 5.18 |
| | | 12 | 10 | 53.35 | 3.55 | 5.4 |
| | | 12 | 20 | 50.73 | 4.39 | 5.06 |
| | | 12 | 50 | 43.80 | 8.18 | 5.10 |
| ML103(pXZ18, pBAD33) | ΔfadD, rc_TE⁺ | 0 | 0 | 67.51 | 24.55 | 10.19 |
| | | 8 | 0 | 64.03 | 18.37 | 10.05 |
| | | 12 | 0 | 63.06 | 21.06 | 9.68 |
| ML103(pXZ18, pBAD33- | ΔfadD, rc_TE⁺, | 8 | 5 | 60.27 | 16.44 | 9.13 |
| prpE) | se_prpE⁺ | 8 | 10 | 59.93 | 18.16 | 9.92 |
| | | 8 | 20 | 57.81 | 18.51 | 9.65 |
| | | 12 | 10 | 51.34 | 13.18 | 7.04 |
| | | 12 | 20 | 51.16 | 13.1 | 7.17 |
| | | 12 | 50 | 44.19 | 13.67 | 7.19 |

uc_TE⁺: overexpression of acyl-ACP thioesterase from Umbellularia californica;
rc_TE⁺: overexpression of acyl-ACP thioesterase from Ricinus communis;
se_prpE⁺: overexpression of propionyl-CoA synthetase from Salmonella enterica.

TABLE 4
Concentration of free fatty acid production at 48 h

| | | Culture Condition | | | | | | | | | | | | |
|---|---|---|---|---|---|---|---|---|---|---|---|---|---|---|
| | Relevant | Propionate | Arabinose | Concentrations of free fatty acid (g/L) | | | | | | | | | | |
| Strain | genotype | (mM) | (mM) | C10 | C11 | C12:1 | C12 | C13 | C14:1 | C14 | C15 | C16:1 | C16 | C18 |
| ML103 | ΔfadD, | 0 | 0 | 0.016 | 0 | 0.18 | 1.22 | 0 | 0.13 | 0.076 | 0 | 0.02 | 0.015 | 0.038 |
| (pXZM12, | uc_TE⁺ | 8 | 0 | 0.013 | 0.017 | 0.16 | 0.93 | 0.028 | 0.11 | 0.055 | 0.0017 | 0.011 | 0.0096 | 0.033 |
| pBAD33) | | 12 | 0 | 0.0068 | 0.048 | 0.12 | 0.42 | 0.08 | 0.077 | 0.027 | 0.0044 | 0.012 | 0.0073 | 0.022 |
| ML103 | ΔfadD, | 8 | 5 | 0.010 | 0.044 | 0.16 | 0.82 | 0.072 | 0.11 | 0.051 | 0.0035 | 0.012 | 0.0084 | 0.032 |
| (pXZM12, | uc_TE⁺, | 8 | 10 | 0.010 | 0.054 | 0.16 | 0.78 | 0.092 | 0.11 | 0.049 | 0.0047 | 0.012 | 0.0090 | 0.033 |
| pBAD33- | se_prpE⁺ | 8 | 20 | 0.0096 | 0.067 | 0.16 | 0.78 | 0.12 | 0.11 | 0.050 | 0.0061 | 0.012 | 0.0096 | 0.032 |
| prpE) | | 12 | 10 | 0.0073 | 0.092 | 0.14 | 0.60 | 0.17 | 0.10 | 0.042 | 0.0088 | 0.013 | 0.0077 | 0.027 |
| | | 12 | 20 | 0.0071 | 0.088 | 0.13 | 0.55 | 0.17 | 0.096 | 0.040 | 0.0091 | 0.011 | 0.0073 | 0.028 |
| | | 12 | 50 | 0.0058 | 0.06 | 0.094 | 0.49 | 0.11 | 0.074 | 0.036 | 0.0064 | 0.010 | 0.010 | 0.027 |
| ML103 | ΔfadD, | 0 | 0 | 0 | 0 | 0 | 0 | 0 | 0 | 0.47 | 0 | 0.41 | 0.67 | 0.27 |
| (pXZ18, | rc_TE⁺ | 8 | 0 | 0 | 0 | 0 | 0 | 0 | 0 | 0.67 | 0.045 | 0.60 | 0.5 | 0.2 |
| (pBADD33) | | 12 | 0 | 0 | 0 | 0 | 0 | 0.0031 | 0 | 0.6 | 0.07 | 0.56 | 0.48 | 0.28 |
| ML103 | ΔfadD, | 8 | 5 | 0 | 0 | 0 | 0 | 0.0053 | 0 | 0.66 | 0.13 | 0.58 | 0.42 | 0.19 |
| (pXZ18, | rc_TE⁺, | 8 | 10 | 0 | 0 | 0 | 0 | 0.0061 | 0 | 0.67 | 0.13 | 0.58 | 0.44 | 0.21 |
| pBAD33- | se_prpE⁺ | 8 | 20 | 0 | 0 | 0 | 0 | 0.0076 | 0 | 0.65 | 0.18 | 0.56 | 0.41 | 0.19 |
| prpE) | | 12 | 10 | 0 | 0 | 0 | 0 | 0.019 | 0 | 0.61 | 0.24 | 0.54 | 0.36 | 0.14 |
| | | 12 | 20 | 0 | 0 | 0 | 0 | 0.02 | 0 | 0.54 | 0.28 | 0.46 | 0.28 | 0.1 |
| | | 12 | 50 | 0 | 0 | 0 | 0 | 0.017 | 0 | 0.45 | 0.2 | 0.36 | 0.23 | 0.073 |

uc_TE⁺: overexpression of acyl-ACP thioesterase from Umbellularia californica;
rc_TE⁺: overexpression of acyl-ACP thioesterase from Ricinus communis;
se_prpE⁺: overexpression of propionyl-CoA synthetase from Salmonella enterica.

TABLE 5
Total fatty acid production and percentage of odd-chain fatty acid production at 48 hr

| | | | | Total odd- | Total even- | | |
|---|---|---|---|---|---|---|---|
| | Relevant | Culture Condition | | chain fatty | chain fatty | Total fatty | Percentage of odd- |
| Strain | genotype | Propionate (mM) | Arabinose (mM) | acid (g/L) | acid (g/L) | acid (g/L) | chain fatty acid (%) |
| ML103(pXZM12, | ΔfadD, uc_TE⁺ | 0 | 0 | 0 | 1.69 | 1.69 | 0 |
| pBAD33) | | 8 | 0 | 0.047 | 1.33 | 1.38 | 3.44 |
| | | 12 | 0 | 0.18 | 1.21 | 1.38 | 12.62 |
| ML103(pXZM12, | ΔfadD, uc_TE⁺, | 8 | 5 | 0.12 | 1.19 | 1.31 | 9.07 |
| pBAD33-prpE) | se_prpE⁺ | 8 | 10 | 0.15 | 1.16 | 1.32 | 11.49 |
| | | 8 | 20 | 0.19 | 1.15 | 1.34 | 14.26 |
| | | 12 | 10 | 0.28 | 0.89 | 1.17 | 23.43 |
| | | 12 | 20 | 0.26 | 0.87 | 1.13 | 23.17 |
| | | 12 | 50 | 0.18 | 0.75 | 0.93 | 19.07 |
| ML103(pXZ18, | ΔfadD, rc_TE⁺ | 0 | 0 | 0 | 1.82 | 1.82 | 0 |
| pBAD33) | | 8 | 0 | 0.045 | 1.97 | 2.01 | 2.25 |
| | | 12 | 0 | 0.073 | 1.91 | 1.99 | 3.68 |
| ML103(pXZ18, | ΔfadD, rc_TE⁺, | 8 | 5 | 0.14 | 1.84 | 1.98 | 6.91 |
| pBAD33-prpE) | se_prpE⁺ | 8 | 10 | 0.14 | 1.9 | 2.04 | 6.91 |
| | | 8 | 20 | 0.18 | 1.81 | 1.99 | 9.19 |
| | | 12 | 10 | 0.25 | 1.65 | 1.9 | 13.34 |
| | | 12 | 20 | 0.3 | 1.38 | 1.68 | 17.68 |
| | | 12 | 50 | 0.22 | 1.1 | 1.32 | 16.66 |

uc_TE⁺: overexpression of acyl-ACP thioesterase from Umbellularia californica;
rc_TE⁺: overexpression of acyl-ACP thioesterase from Ricinus communis;
se_prpE⁺: overexpression of propionyl-CoA synthetase from Salmonella enterica.

TABLE 6
Total glucose consumption and acetate accumulation at 48 hr

| | | Culture Condition | | Glucose consumption | Acetate accumulation | |
|---|---|---|---|---|---|---|
| Strain | Relevant genotype | Propionate (mM) | Arabinose (mM) | (mM) | (mM) | OD₆₀₀ |
| ML103(pXZM12, | ΔfadD, uc_TE⁺ | 0 | 0 | 82.3 | 6.58 | 5.46 |
| pBAD33) | | 8 | 0 | 76.58 | 0 | 3.81 |
| | | 12 | 0 | 80.85 | 0.80 | 2.89 |
| ML103(pXZM12, | ΔfadD, uc_TE⁺, | 8 | 5 | 74.36 | 10.99 | 4.91 |
| pBAD33-prpE) | se_prpE⁺ | 8 | 10 | 75.47 | 12.97 | 4.61 |
| | | 8 | 20 | 75.30 | 15.55 | 5.25 |
| | | 12 | 10 | 79.46 | 8.24 | 7.11 |
| | | 12 | 20 | 79.41 | 13.95 | 7.03 |
| | | 12 | 50 | 64.26 | 23.65 | 6.99 |
| ML103(pXZ18, | ΔfadD, rc_TE⁺ | 0 | 0 | 81.13 | 38.24 | 13.05 |
| pBAD33) | | 8 | 0 | 81.39 | 11.68 | 6.21 |
| | | 12 | 0 | 80.45 | 16.28 | 5.62 |
| ML103(pXZ18, | ΔfadD, rc_TE⁺, | 8 | 5 | 81.09 | 16.82 | 6.62 |
| pBAD33-prpE) | se_prpE⁺ | 8 | 10 | 81.8 | 18.11 | 7.01 |
| | | 8 | 20 | 81.52 | 17.34 | 7.02 |
| | | 12 | 10 | 78.72 | 7.17 | 9.56 |
| | | 12 | 20 | 76.35 | 7.43 | 8.24 |
| | | 12 | 50 | 61.77 | 10.30 | 7.35 |

uc_TE⁺: overexpression of acyl-ACP thioesterase from Umbellularia californica;
rc_TE⁺: overexpression of acyl-ACP thioesterase from Ricinus communis;
se_prpE⁺: overexpression of propionyl-CoA synthetase from Salmonella enterica.

TABLE 7
Concentration of free fatty acid production at 24 hr

| | | Culture | | | | | | | | | | | | |
|---|---|---|---|---|---|---|---|---|---|---|---|---|---|---|
| | | Condition | | | | | | | | | | | | |
| | | Propio- | Arabi- | | | | | | | | | | | |
| | Relevant | nate | nose | Concentrations of free fatty acid (g/L) | | | | | | | | | | |
| Strain | genotype | (mM) | (mM) | C10 | C11 | C12:1 | C12 | C13 | C14:1 | C14 | C15 | C16:1 | C16 | C18 |
| ML103ΔfabH | ΔfadD, | 12 | 10 | 0 | 0.0028 | 0.0047 | 0.02 | 0.023 | 0.0084 | 0.0049 | 0.0042 | 0.0048 | 0.012 | 0.017 |
| (pXZM12, pBH1) | ΔfabH, | | | | | | | | | | | | | |
| | uc_TE⁺, | | | | | | | | | | | | | |
| | bs_ fabH | | | | | | | | | | | | | |
| | B⁺ | | | | | | | | | | | | | |
| ML103ΔfabH | ΔfadD, | 12 | 10 | 0 | 0.0068 | 0.0088 | 0.035 | 0.04 | 0.013 | 0.0058 | 0.0057 | 0.0045 | 0.009 | 0.013 |
| (pXZM12, pBH2) | ΔfabH, | | | | | | | | | | | | | |
| | uc_TE⁺, | | | | | | | | | | | | | |
| | sa_fabH⁺ | | | | | | | | | | | | | |
| ML103ΔfabH | ΔfadD, | 12 | 10 | 0 | 0.0048 | 0.0057 | 0.026 | 0.045 | 0.0093 | 0.0062 | 0.0076 | 0.0041 | 0.0093 | 0.01 |
| (pXZM12, pBH3) | ΔfabH, | | | | | | | | | | | | | |
| | uc_TE⁺, | | | | | | | | | | | | | |
| | bs_fabH | | | | | | | | | | | | | |
| | A⁺ | | | | | | | | | | | | | |
| ML103ΔfabH | ΔfadD, | 12 | 10 | 0 | 0 | 0.0012 | 0.012 | 0.0021 | 0.0024 | 0.0068 | 0.00064 | 0.0026 | 0.0088 | 0.01 |
| (pXZM12, pBH4) | ΔfabH, | | | | | | | | | | | | | |
| | uc_TE⁺, | | | | | | | | | | | | | |
| | sp_dpsC⁺ | | | | | | | | | | | | | |
| ML103ΔfabH | ΔfadD, | 12 | 10 | 0 | 0 | 0 | 0 | 0.013 | 0 | 0.34 | 0.42 | 0.37 | 0.27 | 0.21 |
| (pXZ18, pBH1) | ΔfabH, | | | | | | | | | | | | | |
| | rc_TE⁺, | | | | | | | | | | | | | |
| | bs_fabH | | | | | | | | | | | | | |
| | B⁺ | | | | | | | | | | | | | |
| ML103ΔfabH | ΔfadD, | 12 | 10 | 0 | 0 | 0 | 0 | 0.007 | 0 | 0.14 | 0.26 | 0.24 | 0.16 | 0.19 |
| (pXZ18, pBH2) | ΔfabH, | | | | | | | | | | | | | |
| | rc_TE⁺, | | | | | | | | | | | | | |
| | sa_ fabH⁺ | | | | | | | | | | | | | |
| ML103ΔfabH | ΔfadD, | 12 | 10 | 0 | 0 | 0 | 0 | 0.0016 | 0 | 0.037 | 0.081 | 0.056 | 0.081 | 0.06 |
| (pXZ18, pBH3) | ΔfabH, | | | | | | | | | | | | | |
| | rc_TE⁺, | | | | | | | | | | | | | |
| | bs_ fabH | | | | | | | | | | | | | |
| | A⁺ | | | | | | | | | | | | | |
| ML103ΔfabH | ΔfadD, | 12 | 10 | 0 | 0 | 0 | 0 | 0 | 0 | 0.0041 | 0.0015 | 0.011 | 0.023 | 0.03 |
| (pXZ18, pBH4) | ΔfabH, | | | | | | | | | | | | | |
| | uc_TE⁺, | | | | | | | | | | | | | |
| | sp_dpsC⁺ | | | | | | | | | | | | | |

uc_TE⁺: overexpression of acyl-ACP thioesterase from Umbellularia californica;
rc_TE⁺: overexpression of acyl-ACP thioesterase from Ricinus communis;
bs_fabH B⁺: overexpression of β-ketoacyl-ACP synthase III B from Bacillus subtilis;
sa_fabH⁺: overexpression of β-ketoacyl-ACP synthase III B from Staphylococcus aureus;
bs_fabH A⁺: overexpression of β-ketoacyl-ACP synthase III A from Bacillus subtilis;
sp_dpsC⁺: overexpression of β-ketoacyl:acyl carrier protein synthase subunit from Streptomyces peucetius.

TABLE 8
Total fatty acid production and % odd-chain at 24 hr

| | | Culture Condition | | | | | |
|---|---|---|---|---|---|---|---|
| | Relevant | Propionate | Arabinose | Total odd-chain | Total even-chain | Total fatty acid | Percentage of odd-chain |
| Strain | genotype | (mM) | (mM) | fatty acid (g/L) | fatty acid (g/L) | (g/L) | fatty acid (%) |
| ML103ΔfabH | ΔfadD, ΔfabH, | 12 | 10 | 0.03 | 0.071 | 0.1 | 29.97 |
| (pXZM12, pBH1) | uc_TE⁺, bs_ fabH B⁺ | | | | | | |
| ML103ΔfabH | ΔfadD, ΔfabH, | 12 | 10 | 0.053 | 0.088 | 0.14 | 37.28 |
| (pXZM12, pBH2) | uc_TE⁺, sa_ fabH⁺ | | | | | | |
| ML103ΔfabH | ΔfadD, ΔfabH, | 12 | 10 | 0.057 | 0.07 | 0.13 | 44.99 |
| (pXZM12, pBH3) | uc_TE⁺, bs_ fabH A⁺ | | | | | | |
| ML103ΔfabH | ΔfadD, ΔfabH, | 12 | 10 | 0.0027 | 0.044 | 0.047 | 5.71 |
| (pXZM12, pBH4) | uc_TE⁺, sp_dpsC⁺ | | | | | | |
| ML103ΔfabH | ΔfadD, ΔfabH, | 12 | 10 | 0.44 | 1.19 | 1.62 | 26.9 |
| (pXZ18, pBH1) | rc_TE⁺, bs_ fabH B⁺ | | | | | | |
| ML103ΔfabH | ΔfadD, ΔfabH, | 12 | 10 | 0.26 | 0.72 | 0.99 | 26.65 |
| (pXZ18, pBH2) | rc_TE⁺, sa_ fabH⁺ | | | | | | |
| ML103ΔfabH | ΔfadD, ΔfabH, | 12 | 10 | 0.083 | 0.23 | 0.32 | 26.25 |
| (pXZ18, pBH3) | rc_TE⁺, bs_ fabH A⁺ | | | | | | |
| ML103ΔfabH | ΔfadD, ΔfabH, | 12 | 10 | 0.0015 | 0.067 | 0.069 | 2.09 |
| (pXZ18, pBH4) | uc_TE⁺, sp_dpsC⁺ | | | | | | |

uc_TE⁺: overexpression of acyl-ACP thioesterase from Umbellularia californica;
rc_TE⁺: overexpression of acyl-ACP thioesterase from Ricinus communis;
bs_fabH B⁺: overexpression of β-ketoacyl-ACP synthase III B from Bacillus subtilis;
sa_ fabH⁺: overexpression of β-ketoacyl-ACP synthase III B from Staphylococcus aureus;
bs_ fabH A⁺: overexpression of β-ketoacyl-ACP synthase III A from Bacillus subtilis;
sp_dpsC⁺: overexpression of β-ketoacyl:acyl carrier protein synthase subunit (dpsC) from Streptomyces peucetius.

TABLE 9
Total glucose consumption and acetate accumulation at 24 hr

| | | Culture Condition | | | | |
|---|---|---|---|---|---|---|
| | Relevant | Propionate | Arabinose | Glucose | Acetate | |
| Strain | genotype | (mM) | (mM) | consumption (mM) | accumulation (mM) | OD₆₀₀ |
| ML103ΔfabH | ΔfadD, ΔfabH, | 12 | 10 | 32.11 | 13.4 | 4.89 |
| (pXZM12, pBH1) | uc_TE⁺, bs_ fabH B⁺ | | | | | |
| ML103ΔfabH | ΔfadD, ΔfabH, | 12 | 10 | 25.41 | 11.89 | 3.33 |
| (pXZM12, pBH2) | uc_TE⁺, sa_fabH ⁺ | | | | | |
| ML103ΔfabH | ΔfadD, ΔfabH, | 12 | 10 | 32.99 | 10.77 | 4.48 |
| (pXZM12, pBH3) | uc_TE⁺, bs_fabH A⁺ | | | | | |
| ML103ΔfabH | ΔfadD, ΔfabH, | 12 | 10 | 23.79 | 26.95 | 3.21 |
| (pXZM12, pBH4) | uc_TE⁺, sp_dpsC⁺ | | | | | |
| ML103ΔfabH | ΔfadD, ΔfabH, | 12 | 10 | 65.33 | 14.32 | 7.92 |
| (pXZ18, pBH1) | rc_TE⁺, bs_fabH B⁺ | | | | | |
| ML103ΔfabH | ΔfadD, ΔfabH, | 12 | 10 | 48.96 | 8.47 | 6.72 |
| (pXZ18, pBH2) | rc_TE⁺, sa_fabH⁺ | | | | | |
| ML103ΔfabH | ΔfadD, ΔfabH, | 12 | 10 | 37.43 | 14.5 | 5.88 |
| (pXZ18, pBH3) | rc_TE⁺, bs_fabH A⁺ | | | | | |
| ML103ΔfabH | ΔfadD, ΔfabH, | 12 | 10 | 16.81 | 17.49 | 1.78 |
| (pXZ18, pBH4) | uc_TE⁺, sp_dpsC⁺ | | | | | |

uc_TE⁺: overexpression of acyl-ACP thioesterase from Umbellularia californica;
rc_TE⁺: overexpression of acyl-ACP thioesterase from Ricinus communis;
bs_fabH B⁺: overexpression of β-ketoacyl-ACP synthase III B from Bacillus subtilis;
sa_ fabH⁺: overexpression of β-ketoacyl-ACP synthase III B from Staphylococcus aureus;
bs_ fabH A⁺: overexpression of β-ketoacyl-ACP synthase III A from Bacillus subtilis;
sp_dpsC⁺: overexpression of β-ketoacyl:acyl carrier protein synthase subunit (dpsC) from Streptomyces peucetius.

TABLE 10
Concentration of free fatty acid production at 48 hr=

| | | Culture | | | | | | | | | | | | |
|---|---|---|---|---|---|---|---|---|---|---|---|---|---|---|
| | | Condition | | | | | | | | | | | | |
| | | Pro- | Arab- | | | | | | | | | | | |
| | | pio- | i- | | | | | | | | | | | |
| | Relevant | nate | nose | Concentrations of free fatty acid (g/L) | | | | | | | | | | |
| Strain | genotype | (mM) | (mM) | C10 | C11 | C12:1 | C12 | C13 | C14:1 | C14 | C15 | C16:1 | C16 | C18 |
| ML103ΔfabH | ΔfadD, | 12 | 10 | 0 | 0.0025 | 0.0048 | 0.026 | 0.025 | 0.0091 | 0.0064 | 0.0046 | 0.0047 | 0.012 | 0.026 |
| (pXZM12, pBH1) | ΔfabH, | | | | | | | | | | | | | |
| | uc_TE⁺, | | | | | | | | | | | | | |
| | bs_ fabH B⁺ | | | | | | | | | | | | | |
| ML103ΔfabH | ΔfadD, | 12 | 10 | 0 | 0.0092 | 0.01 | 0.048 | 0.051 | 0.015 | 0.0066 | 0.0068 | 0.0046 | 0.01 | 0.013 |
| (pXZM12, pBH2) | ΔfabH, | | | | | | | | | | | | | |
| | uc_TE⁺, | | | | | | | | | | | | | |
| | sa_ fabH⁺ | | | | | | | | | | | | | |
| ML103ΔfabH | ΔfadD, | 12 | 10 | 0 | 0.0072 | 0.0071 | 0.04 | 0.061 | 0.012 | 0.0074 | 0.0091 | 0.0043 | 0.01 | 0.015 |
| (pXZM12, pBH3) | ΔfabH, | | | | | | | | | | | | | |
| | uc_TE⁺, | | | | | | | | | | | | | |
| | bs_ fabH A⁺ | | | | | | | | | | | | | |
| ML103ΔfabH | ΔfadD, | 12 | 10 | 0 | 0 | 0.0015 | 0.019 | 0.0036 | 0.0029 | 0.01 | 0.00054 | 0.0029 | 0.0086 | 0.014 |
| (pXZM12, pBH4) | ΔfabH, | | | | | | | | | | | | | |
| | uc_TE⁺, | | | | | | | | | | | | | |
| | sp_dpsC⁺ | | | | | | | | | | | | | |
| ML103ΔfabH | ΔfadD, | 12 | 10 | 0 | 0 | 0 | 0 | 0.017 | 0 | 0.49 | 0.5 | 0.51 | 0.33 | 0.28 |
| (pXZ18, pBH1) | ΔfabH, | | | | | | | | | | | | | |
| | rc_TE⁺, | | | | | | | | | | | | | |
| | bs_ fabH B⁺ | | | | | | | | | | | | | |
| ML103ΔfabH | ΔfadD, | 12 | 10 | 0 | 0 | 0 | 0 | 0.022 | 0 | 0.37 | 0.44 | 0.47 | 0.23 | 0.28 |
| (pXZ18, pBH2) | ΔfabH, | | | | | | | | | | | | | |
| | rc_TE⁺, | | | | | | | | | | | | | |
| | sa_ fabH⁺ | | | | | | | | | | | | | |
| ML103ΔfabH | ΔfadD, | 12 | 10 | 0 | 0 | 0 | 0 | 0.015 | 0 | 0.34 | 0.35 | 0.47 | 0.24 | 0.31 |
| (pXZ18, pBH3) | ΔfabH, | | | | | | | | | | | | | |
| | rc_TE⁺, | | | | | | | | | | | | | |
| | bs_ fabH A⁺ | | | | | | | | | | | | | |
| ML103ΔfabH | ΔfadD, | 12 | 10 | 0 | 0 | 0 | 0 | 0 | 0 | 0.0074 | 0.0018 | 0.018 | 0.022 | 0.041 |
| (pXZ18, pBH4) | ΔfabH, | | | | | | | | | | | | | |
| | uc_TE⁺, | | | | | | | | | | | | | |
| | sp_dpsC⁺ | | | | | | | | | | | | | |

uc_TE⁺: overexpression of acyl-ACP thioesterase from Umbellularia californica;
rc_TE⁺: overexpression of acyl-ACP thioesterase from Ricinus communis;
bs_fabH B⁺: overexpression of β-ketoacyl-ACP synthase III B from Bacillus subtilis;
sa_fabH⁺: overexpression of β-ketoacyl-ACP synthase III B from Staphylococcus aureus;
bs_ fabH A⁺: overexpression of β-ketoacyl-ACP synthase III A from Bacillus subtilis;
sp_dpsC⁺: overexpression of β-ketoacyl:acyl carrier protein synthase subunit (dpsC) from Streptomyces peucetius.

TABLE 11
Total fatty acid and % odd-chain at 48 h

| | | Culture Condition | | | | | |
|---|---|---|---|---|---|---|---|
| | Relevant | Propionate | Arabinose | Total odd-chain | Total even-chain | Total fatty acid | Percentage of odd-chain |
| Strain | genotype | (mM) | (mM) | fatty acid (g/L) | fatty acid (g/L) | (g/L) | fatty acid (%) |
| ML103ΔfabH | ΔfadD, ΔfabH, | 12 | 10 | 0.032 | 0.088 | 0.12 | 26.91 |
| (pXZM12, pBH1) | uc_TE⁺, bs_ fabH B⁺ | | | | | | |
| ML103ΔfabH | ΔfadD, ΔfabH, | 12 | 10 | 0.067 | 0.11 | 0.17 | 38.45 |
| (pXZM12, pBH2) | uc TE⁺, sa_fabH⁺ | | | | | | |
| ML103ΔfabH | ΔfadD, ΔfabH, | 12 | 10 | 0.078 | 0.097 | 0.17 | 44.51 |
| (pXZM12, pBH3) | uc_TE⁺, bs_ fabH A⁺ | | | | | | |
| ML103ΔfabH | ΔfadD, ΔfabH, | 12 | 10 | 0.0042 | 0.059 | 0.063 | 6.61 |
| (pXZM12, pBH4) | uc_TE⁺, sp_dpsC⁺ | | | | | | |
| ML103ΔfabH | ΔfadD, ΔfabH, | 12 | 10 | 0.52 | 1.62 | 2.13 | 24.26 |
| (pXZ18, pBH1) | rc_TE⁺, bs_ fabH B⁺ | | | | | | |
| ML103ΔfabH | ΔfadD, ΔfabH, | 12 | 10 | 0.46 | 1.36 | 1.82 | 25.28 |
| (pXZ18, pBH2) | rc_TE⁺, sa_ fabH⁺ | | | | | | |
| ML103ΔfabH | ΔfadD, ΔfabH, | 12 | 10 | 0.37 | 1.36 | 1.72 | 21.32 |
| (pXZ18, pBH3) | rc_TE⁺, bs_ fabH A⁺ | | | | | | |
| ML103ΔfabH | ΔfadD, ΔfabH, | 12 | 10 | 0.0018 | 0.089 | 0.091 | 1.98 |
| (pXZ18, pBH4) | uc_TE⁺, sp_dpsC⁺ | | | | | | |

uc_TE⁺: overexpression of acyl-ACP thioesterase from Umbellularia californica;
rc_TE⁺: overexpression of acyl-ACP thioesterase from Ricinus communis;
bs_fabH B⁺: overexpression of β-ketoacyl-ACP synthase III B from Bacillus subtilis;
sa_ fabAH⁺: overexpression of β-ketoacyl-ACP synthase III B from Staphylococcus aureus;
bs_ fabH A⁺: overexpression of β-ketoacyl-ACP synthase III A from Bacillus subtilis;
sp_dpsC⁺: overexpression of β-ketoacyl:acyl carrier protein synthase subunit (dpsC) from Streptomyces peucetius.

TABLE 12
Total glucose consumption and acetate accumulation at 48 hr

| | | Culture Condition | | | | |
|---|---|---|---|---|---|---|
| | Relevant | Propionate | Arabinose | Glucose | Acetate | |
| Strain | genotype | (mM) | (mM) | consumption (mM) | accumulation (mM) | OD₆₀₀ |
| ML103ΔfabH | ΔfadD, ΔfabH, | 12 | 10 | 47.17 | 36.57 | 4.75 |
| (pXZM12, pBH1) | uc_TE⁺, bs_ fabH B⁺ | | | | | |
| ML103ΔfabH | ΔfadD, ΔfabH, | 12 | 10 | 40.09 | 28.11 | 3.54 |
| (pXZM12, pBH2) | uc_TE⁺, sa_ fabH⁺ | | | | | |
| ML103ΔfabH | ΔfadD, ΔfabH, | 12 | 10 | 50.35 | 24.26 | 4.41 |
| (pXZM12, pBH3) | uc_TE⁺, bs_ fabH A⁺ | | | | | |
| ML103ΔfabH | ΔfadD, ΔfabH, | 12 | 10 | 30.88 | 40.18 | 3.25 |
| (pXZM12, pBH4) | uc_TE⁺, sp_dpsC⁺ | | | | | |
| ML103ΔfabH | ΔfadD, ΔfabH, | 12 | 10 | 83.68 | 9.79 | 5.47 |
| (pXZ18, pBH1) | rc_TE⁺, bs_ fabH B⁺ | | | | | |
| ML103ΔfabH | ΔfadD, ΔfabH, | 12 | 10 | 84.46 | 7.05 | 6.1 |
| (pXZ18, pBH2) | rc_TE⁺, sa_ fabH⁺ | | | | | |
| ML103ΔfabH | ΔfadD, ΔfabH, | 12 | 10 | 84.08 | 7.02 | 8.43 |
| (pXZ18, pBH3) | rc_TE⁺, bs_ fabH A⁺ | | | | | |
| ML103ΔfabH | ΔfadD, ΔfabH, | 12 | 10 | 22.66 | 24.54 | 1.72= |
| (pXZ18, pBH4) | uc_TE⁺, sp_dpsC⁺ | | | | | |

uc_TE⁺: overexpression of acyl-ACP thioesterase from Umbellularia californica;
rc_TE⁺: overexpression of acyl-ACP thioesterase from Ricinus communis;
bs_fabH B⁺: overexpression of β-ketoacyl-ACP synthase III B from Bacillus subtilis;
sa_ fabH⁺: overexpression of β-ketoacyl-ACP synthase III B from Staphylococcus aureus;
bs_ fabH A⁺: overexpression of β-ketoacyl-ACP synthase III A from Bacillus subtilis;
sp_dpsC⁺: overexpression of β-ketoacyl:acyl carrier protein synthase subunit (dpsC) from Streptomyces peucetius.

TABLE 13
Concentration of free fatty acid production at 24 hr

| | | Culture Condition | | | | | | | | | | | | | | | | |
|---|---|---|---|---|---|---|---|---|---|---|---|---|---|---|---|---|---|---|
| | Relevant | IPTG | Propionate | Arabinose | Concentrations of free fatty acid (g/L) | | | | | | | | | | | | | |
| Strain | genotype | (mM) | (mM) | (mM) | C7 | C8 | C9 | C10 | C11 | C12:1 | C12 | C13 | C14:1 | C14 | C15 | C16:1 | C16 | C18 |
| ML103 (pXZM12, | ΔfadD, uc_TE⁺, | 0.2 | 12 | 10 | 0 | 0 | 0 | 0.0065 | 0.24 | 0.16 | 0.6 | 0.42 | 0.086 | 0.034 | 0.015 | 0.016 | 0.029 | 0 |
| pBHE1) | se_prpE⁺, bs_ fabH B⁺ | | | | | | | | | | | | | | | | | |
| ML103 (pXZM12, | ΔfadD, uc_TE⁺, | 0.2 | 12 | 10 | 0 | 0 | 0 | 0.0024 | 0.011 | 0.0035 | 0.019 | 0.013 | 0.0028 | 0.0039 | 0.017 | 0.0054 | 0.014 | 0 |
| pBHE2) | se_prpE⁺, sa_ fabH⁺ | | | | | | | | | | | | | | | | | |
| ML103 (pXZM12, | ΔfadD, uc_TE⁺, | 0.2 | 12 | 10 | 0 | 0 | 0 | 0.0014 | 0.079 | 0.043 | 0.25 | 0.21 | 0.038 | 0.015 | 0.0091 | 0.0039 | 0.0056 | 0.0013 |
| pBHE3) | se_prpE⁺, bs_ fabH A⁺ | | | | | | | | | | | | | | | | | |
| ML103 (pXZM12, | ΔfadD, uc_TE⁺, | 0.2 | 12 | 10 | 0 | 0 | 0 | 0.0087 | 0.13 | 0.22 | 0.82 | 0.24 | 0.13 | 0.042 | 0.013 | 0.0099 | 0.0064 | 0.0034 |
| pBHE4) | se_prpE⁺, sp_dpsC⁺ | | | | | | | | | | | | | | | | | |
| ML103 (pXZ18, | ΔfadD, rc_TE⁺, | 0.5 | 12 | 10 | 0 | 0 | 0 | 0 | 0 | 0 | 0 | 0.016 | 0 | 0.63 | 0.33 | 0.42 | 0.37 | 0.026 |
| pBHE1) | se_prpE⁺, bs_ fabH B⁺ | | | | | | | | | | | | | | | | | |
| ML103 (pXZ18, | ΔfadD, rc_TE⁺, | 0.5 | 12 | 10 | 0 | 0 | 0 | 0 | 0 | 0 | 0 | 0.0014 | 0 | 0.045 | 0.059 | 0.015 | 0.12 | 0 |
| pBHE2) | se_prpE⁺, sa_ fabH⁺ | | | | | | | | | | | | | | | | | |
| ML103 (pXZ18, | ΔfadD, rc_TE⁺, | 0.5 | 12 | 10 | 0 | 0 | 0 | 0 | 0 | 0 | 0 | 0.04 | 0 | 0.13 | 0.33 | 0.079 | 0.1 | 0.01 |
| pBHE3) | se_prpE⁺, bs_ fabHA⁺ | | | | | | | | | | | | | | | | | |
| ML103 (pXZ18, | ΔfadD, rc_TE⁺, | 0.5 | 12 | 10 | 0 | 0 | 0 | 0 | 0 | 0 | 0 | 0.0046 | 0 | 0.76 | 0.088 | 0.59 | 0.45 | 0.057 |
| pBHE4) | se_prpE⁺, sp_dpsC⁺ | | | | | | | | | | | | | | | | | |
| ML103ΔfabH | ΔfadD, ΔfabH, | 0.2 | 12 | 10 | 0 | 0 | 0 | 0.0026 | 0.11 | 0.07 | 0.21 | 0.23 | 0.055 | 0.018 | 0.02 | 0.0071 | 0.0087 | 0.0054 |
| (pXZM12, pBHE1) | uc_TE⁺, se_prpE⁺, | | | | | | | | | | | | | | | | | |
| | bs_ fabH B⁺ | | | | | | | | | | | | | | | | | |
| ML103ΔfabH | ΔfadD, ΔfabH, | 0.2 | 12 | 10 | 0 | 0 | 0 | 0.0011 | 0.34 | 0.042 | 0.12 | 0.5 | 0.033 | 0.01 | 0.027 | 0.0054 | 0.0073 | 0.0027 |
| (pXZM12, pBHE2) | uc_TE⁺, se_prpE⁺, | | | | | | | | | | | | | | | | | |
| | sa_ fabH⁺ | | | | | | | | | | | | | | | | | |
| ML103ΔfabH | ΔfadD, ΔfabH, | 0.2 | 12 | 10 | 0 | 0 | 0 | 0 | 0.015 | 0.0047 | 0.02 | 0.15 | 0.0083 | 0.0044 | 0.017 | 0.003 | 0.0063 | 0 |
| (pXZM12, pBHE3) | uc_TE⁺, se_prpE⁺, | | | | | | | | | | | | | | | | | |
| | bs_ fabHA⁺ | | | | | | | | | | | | | | | | | |
| ML103ΔfabH | ΔfadD, ΔfabH, | 0.2 | 12 | 10 | 0 | 0 | 0 | 0 | 0.00047 | 0.002 | 0.015 | 0.0025 | 0.0039 | 0.002 | 0.0027 | 0.0021 | 0.0036 | 0 |
| (pXZM12, pBHE4) | uc_TE⁺, se_prpE⁺, | | | | | | | | | | | | | | | | | |
| | sp_dpsC⁺ | | | | | | | | | | | | | | | | | |
| ML103ΔfabH | ΔfadD, ΔfabH, | 0.5 | 12 | 10 | 0 | 0 | 0 | 0 | 0 | 0 | 0 | 0.028 | 0 | 0.4 | 0.62 | 0.32 | 0.29 | 0.052 |
| (pXZ18, pBHE1) | rc_TE⁺, se_prpE⁺, | | | | | | | | | | | | | | | | | |
| | bs_ fabH B⁺ | | | | | | | | | | | | | | | | | |
| ML103ΔfabH | ΔfadD, rc_TE⁺, | 0.5 | 12 | 10 | 0 | 0 | 0 | 0 | 0 | 0 | 0 | 0.091 | 0 | 0.19 | 0.73 | 0.15 | 0.14 | 0.032 |
| (pXZ18, pBHE2) | se_prpE⁺, sa_ fabH⁺ | | | | | | | | | | | | | | | | | |
| ML103ΔfabH | ΔfadD, ΔfabH, | 0.5 | 12 | 10 | 0 | 0 | 0 | 0 | 0 | 0 | 0 | 0.05 | 0 | 0.028 | 0.57 | 0.03 | 0.042 | 0.0088 |
| (pXZ18, pBHE3) | rc_TE⁺, se_prpE⁺, | | | | | | | | | | | | | | | | | |
| | bs_fabHA⁺ | | | | | | | | | | | | | | | | | |
| ML103ΔfabH | ΔfadD, ΔfabH, | 0.5 | 12 | 10 | 0 | 0 | 0 | 0 | 0 | 0 | 0 | 0 | 0 | 0.0034 | 0.0025 | 0.0098 | 0.018 | 0.0043 |
| (pXZ18, pBHE4) | rc_TE⁺, se_prpE⁺, | | | | | | | | | | | | | | | | | |
| | sp_dpsC⁺ | | | | | | | | | | | | | | | | | |
| ML191Δfabh | ΔpfkA, ΔfadD, | 0.1 | 12 | 10 | 0.11 | 0.097 | 0.26 | 0 | 0 | 0 | 0 | 0 | 0 | 0 | 0 | 0 | 0 | 0 |
| (pXZmch, pBHE2) | ΔfabH, ch_TE⁺, | 0.5 | 12 | 10 | 0.064 | 0.014 | 0.11 | 0 | 0 | 0 | 0 | 0 | 0 | 0 | 0 | 0 | 0 | 0 |
| | se_prpE⁺, sa_ fabH⁺ | 0.5 | 12 | 20 | 0 | 0 | 0 | 0 | 0 | 0 | 0 | 0 | 0 | 0 | 0 | 0 | 0 | 0 |

uc_TE⁺: overexpression of acyl-ACP thioesterase from U. californica;
rc_TE⁺: overexpression of acyl-ACP thioesterase from R. communis;
ch_TE⁺: overexpression of acyl-ACP thioesterase from C. hookeriana;
se_prpE⁺: overexpression of propionyl-CoA synthetase from S. enterica;
bs_fabH B⁺: overexpression of β-ketoacyl-ACP synthase III B from B. subtilis;
sa_ fabH⁺: overexpression of β-ketoacyl-ACP synthase III B from S. aureus;
bs_ fabH A⁺: overexpression of β-ketoacyl-ACP synthase III A from B. subtilis;
sp_dpsC⁺: overexpression of β-ketoacyl:acyl carrier protein synthase subunit (dpsC) from S. peucetius.

TABLE 14
Total fatty acid production and percentage of odd-chain fatty acid production at 24 hr

| | | | | | Total | Total | Total | Percentage |
|---|---|---|---|---|---|---|---|---|
| | | Culture Condition | | | odd-chain | even-chain | fatty | of odd-chain |
| | Relevant | IPTG | Propionate | Arabinose | fatty acid | fatty acid | acid | fatty |
| Strain | genotype | (mM) | (mM) | (mM) | (g/L) | (g/L) | (g/L) | acid (%) |
| ML103 | ΔfadD, uc_TE⁺, | 0.2 | 12 | 10 | 0.68 | 0.93 | 1.61 | 42.39 |
| (pXZM12, pBHE1) | se_prpE⁺, bs_fabH B⁺ | | | | | | | |
| ML103 | ΔfadD, uc_TE⁺, | 0.2 | 12 | 10 | 0.041 | 0.051 | 0.092 | 44.4 |
| (pXZM12, pBHE2) | se_prpE⁺, sa_ fabH⁺ | | | | | | | |
| ML103 | ΔfadD, uc_TE⁺, | 0.2 | 12 | 10 | 0.29 | 0.36 | 0.65 | 44.97 |
| (pXZM12, pBHE3) | se_prpE⁺, bs_ fabH A⁺ | | | | | | | |
| ML103 | ΔfadD, uc_TE⁺, | 0.2 | 12 | 10 | 0.38 | 1.23 | 1.62 | 23.82 |
| (pXZM12, pBHE4) | se_prpE⁺, sp_dpsC⁺ | | | | | | | |
| ML103 | ΔfadD, rc_TE⁺, | 0.5 | 12 | 10 | 0.34 | 1.45 | 1.8 | 19.1 |
| (pXZ18, pBHE1) | se_prpE⁺, bs_ fabH B⁺ | | | | | | | |
| ML103 | Δfad, rc_TE⁺, | 0.5 | 12 | 10 | 0.06 | 0.18 | 0.24 | 25.43 |
| (pXZ18, pBHE2) | se_prpE⁺, sa_ fabH⁺ | | | | | | | |
| ML103 | ΔfadD, rc_TE⁺, | 0.5 | 12 | 10 | 0.37 | 0.32 | 0.69 | 53.17 |
| (pXZ18, pBHE3) | se_prpE⁺, bs_ fabH A⁺ | | | | | | | |
| ML103 | ΔfadD, rc_TE⁺, | 0.5 | 12 | 10 | 0.092 | 1.85 | 1.95 | 4.81 |
| (pXZ18, pBHE4) | se_prpE⁺, sp_dpsC⁺ | | | | | | | |
| ML103ΔfabH | ΔfadD, ΔfabH, | 0.2 | 12 | 10 | 0.37 | 0.38 | 0.75 | 49.29 |
| (pXZM12, pBHE1) | uc_TE⁺, se_prpE⁺, | | | | | | | |
| | bs_ fabH B⁺ | | | | | | | |
| ML103ΔfabH | ΔfadD, ΔfabH, | 0.2 | 12 | 10 | 0.87 | 0.23 | 1.1 | 79.4 |
| (pXZM12, pBHE2) | uc_TE⁺, se_prpE⁺, | | | | | | | |
| | sa_ fabH⁺ | | | | | | | |
| ML103ΔfabH | ΔfadD, ΔfabH, | 0.2 | 12 | 10 | 0.18 | 0.046 | 0.22 | 79.28 |
| (pXZM12, pBHE3) | uc_TE⁺, se_prpE⁺, | | | | | | | |
| | bs_ fabH A⁺ | | | | | | | |
| ML103ΔfabH | ΔfadD, ΔfabH, | 0.2 | 12 | 10 | 0.0057 | 0.029 | 0.035 | 16.05 |
| (pXZM12, pBHE4) | uc_TE⁺, se_prpE⁺, | | | | | | | |
| | sp_dpsC⁺ | | | | | | | |
| ML103ΔfabH | ΔfadD, ΔfabH, | 0.5 | 12 | 10 | 0.64 | 1.06 | 1.71 | 37.76 |
| (pXZ18, pBHE1) | rc_TE⁺, se_prpE⁺, | | | | | | | |
| | bs_ fabH B⁺ | | | | | | | |
| ML103ΔfabH | ΔfadD, rc_TE⁺, | 0.5 | 12 | 10 | 0.82 | 0.51 | 1.33 | 61.68 |
| (pXZ18, pBHE2) | se_prpE⁺, sa_ fabH⁺ | | | | | | | |
| ML103ΔfabH | ΔfadD, ΔfabH, | 0.5 | 12 | 10 | 0.62 | 0.11 | 0.73 | 85.11 |
| (pXZ18, pBHE3) | rc_TE⁺, se_prpE⁺, | | | | | | | |
| | bs_fabH A⁺ | | | | | | | |
| ML103ΔfabH | ΔfadD, ΔfabH, | 0.5 | 12 | 10 | 0.0025 | 0.035 | 0.037 | 6.53 |
| (pXZ18, pBHE4) | rc_TE⁺, se_prpE⁺, | | | | | | | |
| | sp_dpsC⁺ | | | | | | | |
| ML191ΔfabH | ΔpfkA, ΔfadD, | 0.1 | 12 | 10 | 0.38 | 0.0978 | 0.47 | 80.85 |
| (pXZmch, pBHE2) | ΔfabH, ch_TE⁺, | 0.5 | 12 | 10 | 0.178 | 0.0148 | 0.192 | 92.7 |
| | se_prpE⁺, sa_ fabH⁺ | 0.5 | 12 | 20 | 0 | 0 | 0 | 0 |

uc_TE⁺: overexpression of acyl-ACP thioesterase from U. californica;
rc_TE⁺: overexpression of acyl-ACP thioesterase from R. communis;
ch_TE+: overexpression of acyl-ACP thioesterase from C. hookeriana;
se_prpE⁺: overexpression of propionyl-CoA synthetase from S. enterica;
bs_fabH B⁺: overexpression of β-ketoacyl-ACP synthase III B from B. subtilis;
sa_ fabH⁺: overexpression of β-ketoacyl-ACP synthase III B from S. aureus;
bs_ fabH A⁺: overexpression of β-ketoacyl-ACP synthase III A from B. subtilis;
sp_dpsC⁺: overexpression of β-ketoacyl:acyl carrier protein synthase subunit (dpsC) from S. peucetius.

TABLE 15
Total glucose consumption and acetate accumulation at 24 hr

| | | Culture Condition | | | Glucose | Acetate | |
|---|---|---|---|---|---|---|---|
| | Relevant | IPTG | Propionate | Arabinose | consumption | accumulation | |
| Strain | genotype | (mM) | (mM) | (mM) | (mM) | (mM) | OD₆₀₀ |
| ML103 | ΔfadD, uc_TE⁺, | 0.2 | 12 | 10 | 51.7 | 2.25 | 5.84 |
| (pXZM12, pBHE1) | se_prpE⁺, bs_ fabH B⁺ | | | | | | |
| ML103 | ΔfadD, uc_TE⁺, | 0.2 | 12 | 10 | 12.36 | 16.46 | 3.13 |
| (pXZM12, pBHE2) | se_prpE⁺, sa_ fabH⁺ | | | | | | |
| ML103 | ΔfadD, uc_TE⁺, | 0.2 | 12 | 10 | 29.2 | 20.29 | 4.62 |
| (pXZM12, pBHE3) | se_prpE⁺, bs_ fabH A⁺ | | | | | | |
| ML103 | ΔfadD, uc_TE⁺, | 0.2 | 12 | 10 | 54.94 | 1.65 | 5.92 |
| (pXZM12, pBHE4) | se_prpE⁺, sp_dpsC⁺ | | | | | | |
| ML103 | ΔfadD, rc_TE⁺, | 0.5 | 12 | 10 | 57.03 | 21.69 | 8.96 |
| (pXZ18, pBHE1) | se_prpE⁺, bs_ fabH B⁺ | | | | | | |
| ML103 | ΔfadD, rc_TE⁺, | 0.5 | 12 | 10 | 24.83 | 37.71 | 4.79 |
| (pXZ18, pBHE2) | se_prpE⁺, sa_ fabH⁺ | | | | | | |
| ML103 | ΔfadD, rc_TE⁺, | 0.5 | 12 | 10 | 36.44 | 24.34 | 7.57 |
| (pXZ18, pBHE3) | se_prpE⁺, bs_ fabH A⁺ | | | | | | |
| ML103 | ΔfadD, rc_TE⁺, | 0.5 | 12 | 10 | 58.6 | 17.48 | 10.03 |
| (pXZ18, pBHE4) | se_prpE⁺, sp_dpsC⁺ | | | | | | |
| ML103ΔfabH | ΔfadD, ΔfabH, | 0.2 | 12 | 10 | 39.6 | 8.87 | 5.37 |
| (pXZM12, pBHE1) | uc_TE⁺, se_prpE⁺, | | | | | | |
| | bs_ fabH B⁺ | | | | | | |
| ML103ΔfabH | ΔfadD, ΔfabH, | 0.2 | 12 | 10 | 42.55 | 13.25 | 4.97 |
| (pXZM12, pBHE2) | uc_TE⁺, se_prpE⁺, | | | | | | |
| | sa_ fabH⁺ | | | | | | |
| ML103ΔfabH | ΔfadD, ΔfabH, | 0.2 | 12 | 10 | 24.38 | 14.3 | 4.13 |
| (pXZM12, pBHE3) | uc_TE⁺, se_prpE⁺, | | | | | | |
| | bs_ fabH A⁺ | | | | | | |
| ML103ΔfabH | ΔfadD, ΔfabH, | 0.2 | 12 | 10 | 16.5 | 26.59 | 2.43 |
| (pXZM12, pBHE4) | uc_TE⁺, se_prpE⁺, | | | | | | |
| | sp_dpsC⁺ | | | | | | |
| ML103ΔfabH | ΔfadD, ΔfabH, | 0.5 | 12 | 10 | 57.28 | 20.19 | 8.57 |
| (pXZ18, pBHE1) | rc_TE⁺, se_prpE⁺, | | | | | | |
| | bs_ fabH B⁺ | | | | | | |
| ML103ΔfabH | ΔfadD, rc_TE⁺, | 0.5 | 12 | 10 | 45.94 | 17.43 | 8.26 |
| (pXZ18, pBHE2) | se_prpE⁺, sa_ fabH⁺ | | | | | | |
| ML103ΔfabH | ΔfadD, ΔfabH, | 0.5 | 12 | 10 | 33.05 | 20.77 | 7.69 |
| (pXZ18, pBHE3) | rc_TE⁺, se_prpE⁺, | | | | | | |
| | bs_ fabH A⁺ | | | | | | |
| ML103ΔfabH | ΔfadD, ΔfabH, | 0.5 | 12 | 10 | 5.44 | 18.15 | 1.36 |
| (pXZ18, pBHE4) | rc_TE⁺, se_prpE⁺, | | | | | | |
| | sp_dpsC⁺ | | | | | | |
| ML191ΔfabH | ΔpfkA, ΔfadD, | 0.1 | 12 | 10 | 41.69 | 10.51 | 6.16 |
| (pXZmch, pBHE2) | ΔfabH, ch_TE⁺, | 0.5 | 12 | 10 | 12.42 | 13.51 | 3.14 |
| | se_prpE⁺, sa_ fabH⁺ | 0.5 | 12 | 20 | 12.64 | 14.92 | 2.22 |

uc_TE⁺: overexpression of acyl-ACP thioesterase from U. californica;
rc_TE⁺: overexpression of acyl-ACP thioesterase from R. communis;
ch_TE+: overexpression of acyl-ACP thioesterase from C. hookeriana;
se_prpE⁺: overexpression of propionyl-CoA synthetase from S. enterica;
bs_fabH B⁺: overexpression of β-ketoacyl-ACP synthase III B from B. subtilis;
sa_ fabH⁺: overexpression of β-ketoacyl-ACP synthase III B from S. aureus;
bs_ fabH A⁺: overexpression of β-ketoacyl-ACP synthase III A from B. subtilis;
sp_dpsC⁺: overexpression of β-ketoacyl:acyl carrier protein synthase subunit (dpsC) from S. peucetius.

TABLE 16
Concentration of free fatty acid production at 48 hr

| | | Culture Condition | | | | | | | | | | | | | | | | |
|---|---|---|---|---|---|---|---|---|---|---|---|---|---|---|---|---|---|---|
| | Relevant | IPTG | Propionate | Arabinose | Concentrations of free fatty acid (g/L) | | | | | | | | | | | | | |
| Strain | genotype | (mM) | (mM) | (mM) | C7 | C8 | C9 | C10 | C11 | C12:1 | C12 | C13 | C14:1 | C14 | C15 | C16:1 | C16 | C18 |
| ML103 | ΔfadD, uc_TE⁺, se_prpE⁺, | 0.2 | 12 | 10 | 0 | 0 | 0 | 0.01 | 0.33 | 0.26 | 0.99 | 0.63 | 0.16 | 0.069 | 0.032 | 0.028 | 0.025 | 0.0048 |
| (pXZM12, pBHE1) | bs_ fabH B⁺ | | | | | | | | | | | | | | | | | |
| ML103 | ΔfadD, uc_TE⁺, se_prpE⁺, | 0.2 | 12 | 10 | 0 | 0 | 0 | 0.0084 | 0.012 | 0.0046 | 0.032 | 0.02 | 0.0051 | 0.0086 | 0.018 | 0.0077 | 0.021 | 0.0038 |
| (pXZM12, pBHE2) | sa_ fabH⁺ | | | | | | | | | | | | | | | | | |
| ML103 | ΔfadD, uc_TE⁺, se_prpE⁺, | 0.2 | 12 | 10 | 0 | 0 | 0 | 0.0017 | 0.079 | 0.043 | 0.28 | 0.23 | 0.043 | 0.02 | 0.013 | 0.0071 | 0.011 | 0.0017 |
| (pXZM12, pBHE3) | bs_ fabH A⁺ | | | | | | | | | | | | | | | | | |
| ML103 | ΔfadD, uc_TE⁺, se_prpE⁺, | 0.2 | 12 | 10 | 0 | 0 | 0 | 0.015 | 0.17 | 0.32 | 1.24 | 0.27 | 0.19 | 0.066 | 0.015 | 0.019 | 0.0089 | 0.0048 |
| (pXZM12, pBHE4) | sp_dpsC⁺ | | | | | | | | | | | | | | | | | |
| ML103 | ΔfadD, rc_TE⁺, se_prpE⁺, | 0.5 | 12 | 10 | 0 | 0 | 0 | 0 | 0 | 0 | 0 | 0.035 | 0 | 0.98 | 0.53 | 0.63 | 0.51 | 0.041 |
| (pXZ18, pBHE1) | bs_ fabH B⁺ | | | | | | | | | | | | | | | | | |
| ML103 | ΔfadD, rc_T E⁺, se_prpE⁺, | 0.5 | 12 | 10 | 0 | 0 | 0 | 0 | 0 | 0 | 0 | 0.0027 | 0 | 0.062 | 0.069 | 0.017 | 0.13 | 0 |
| (pXZ18, pBHE2) | sa_ fabH⁺ | | | | | | | | | | | | | | | | | |
| ML103 | ΔfadD, rc_TE⁺, se_prpE⁺, | 0.5 | 12 | 10 | 0 | 0 | 0 | 0 | 0 | 0 | 0 | 0.039 | 0 | 0.14 | 0.35 | 0.072 | 0.12 | 0.013 |
| (pXZ18, pBHE3) | bs_ fabH A⁺ | | | | | | | | | | | | | | | | | |
| ML103 | ΔfadD, rc_TE⁺, se_prpE⁺, | 0.5 | 12 | 10 | 0 | 0 | 0 | 0 | 0 | 0 | 0 | 0.013 | 0 | 0.98 | 0.15 | 0.77 | 0.51 | 0.073 |
| (pXZ18, pBHE4) | sp_dpsC⁺ | | | | | | | | | | | | | | | | | |
| ML103ΔfabH | ΔfadD, ΔfabH, uc_TE⁺, | 0.2 | 12 | 10 | 0 | 0 | 0 | 0.0046 | 0.18 | 0.12 | 0.4 | 0.35 | 0.091 | 0.026 | 0.026 | 0.011 | 0.011 | 0.0073 |
| (pXZM12, pBHE1) | se_prpE⁺, bs_ fabH B⁺ | | | | | | | | | | | | | | | | | |
| ML103ΔfabH | ΔfadD, ΔfabH, uc_TE⁺, | 0.2 | 12 | 10 | 0 | 0 | 0 | 0.019 | 0.41 | 0.063 | 0.2 | 0.56 | 0.048 | 0.015 | 0.029 | 0.0065 | 0.0088 | 0.0043 |
| (pXZM12, pBHE2) | se_prpE⁺, sa_ fabH⁺ | | | | | | | | | | | | | | | | | |
| ML103ΔfabH | ΔfadD, ΔfabH, uc_TE⁺, | 0.2 | 12 | 10 | 0 | 0 | 0 | 0 | 0.02 | 0.0052 | 0.02 | 0.14 | 0.0086 | 0.0052 | 0.018 | 0.0036 | 0.0058 | 0 |
| (pXZM12, pBHE3) | se_prpE⁺, bs_ fabH A⁺ | | | | | | | | | | | | | | | | | |
| ML103ΔfabH | ΔfadD, ΔfabH, uc_TE⁺, | 0.2 | 12 | 10 | 0 | 0 | 0 | 0 | 0.00067 | 0.0017 | 0.023 | 0.00087 | 0.006 | 0.022 | 0.025 | 0.013 | 0.029 | 0.0044 |
| (pXZM12, pBHE4) | se_prpE⁺, sp_dpsC⁺ | | | | | | | | | | | | | | | | | |
| ML103ΔfabH | ΔfadD, ΔfabH, rc_TE⁺, | 0.5 | 12 | 10 | 0 | 0 | 0 | 0 | 0 | 0 | 0 | 0.045 | 0 | 0.57 | 0.77 | 0.44 | 0.38 | 0.076 |
| (pXZ18, pBHE1) | se_prpE⁺, bs_ fabH B⁺ | | | | | | | | | | | | | | | | | |
| ML103ΔfabH | ΔfadD, rc_T E⁺, se_prpE⁺, | 0.5 | 12 | 10 | 0 | 0 | 0 | 0 | 0 | 0 | 0 | 0.18 | 0 | 0.37 | 1.02 | 0.21 | 0.17 | 0.041 |
| (pXZ18, pBHE2) | sa_ fabH⁺ | | | | | | | | | | | | | | | | | |
| ML103ΔfabH | ΔfadD, ΔfabH, rc_TE⁺, | 0.5 | 12 | 10 | 0 | 0 | 0 | 0 | 0 | 0 | 0 | 0.045 | 0 | 0.03 | 0.56 | 0.023 | 0.043 | 0.027 |
| (pXZ18, pBHE3) | se_prpE⁺, bs_ fabH A⁺ | | | | | | | | | | | | | | | | | |
| ML103ΔfabH | ΔfadD, ΔfabH, rc_TE⁺, | 0.5 | 12 | 10 | 0 | 0 | 0 | 0 | 0 | 0 | 0 | 0 | 0 | 0.0078 | 0.004 | 0.017 | 0.03 | 0.01 |
| (pXZ18, pBHE4) | se_prpE⁺, sp_dpsC⁺ | | | | | | | | | | | | | | | | | |
| ML191ΔfabH | ΔpfkA, ΔfadD, ΔfabH, | 0.1 | 12 | 10 | 0.37 | 0.13 | 0.46 | 0 | 0 | 0 | 0 | 0 | 0 | 0 | 0 | 0 | 0 | 0 |
| (pXZ mch, pBHE 2) | ch_TE⁺, se_prpE⁺, sa_ fabH⁺ | 0.5 | 12 | 10 | 0.23 | 0.059 | 0.51 | 0 | 0 | 0 | 0 | 0 | 0 | 0 | 0 | 0 | 0 | 0 |
| | | 0.5 | 12 | 20 | 0.21 | 0.090 | 0.39 | 0 | 0 | 0 | 0 | 0 | 0 | 0 | 0 | 0 | 0 | 0 |

uc_TE⁺: overexpression of acyl-ACP thioesterase from U. californica;
rc_TE⁺: overexpression of acyl-ACP thioesterase from R. communis;
ch_TE+: overexpression of acyl-ACP thioesterase from C. hookeriana;
se_prpE⁺: overexpression of propionyl-CoA synthetase from S. enterica;
bs_fabH B⁺: overexpression of β-ketoacyl-ACP synthase III B from B. subtilis;
sa_ fabH⁺: overexpression of β-ketoacyl-ACP synthase III B from S. aureus;
bs_ fabH A⁺: overexpression of β-ketoacyl-ACP synthase III A from B. subtilis;
sp_dpsC⁺: overexpression of β-ketoacyl:acyl carrier protein synthase subunit (dpsC) from S. peucetius.

TABLE 17
Total fatty acid production and percentage of odd-chain fatty acid production at 48 hr

| | | | | | Total | Total | Total | Percentage |
|---|---|---|---|---|---|---|---|---|
| | | Culture Condition | | | odd-chain | even-chain | fatty | of odd-chain |
| | Relevant | IPTG | Propionate | Arabinose | fatty acid | fatty acid | acid | fatty |
| Strain | genotype | (mM) | (mM) | (mM) | (g/L) | (g/L) | (g/L) | acid (%) |
| ML103 | ΔfadD, uc_TE⁺, | 0.2 | 12 | 10 | 0.99 | 1.55 | 2.54 | 39.05 |
| (pXZM12, pBHE1) | se_prpE⁺, bs_ fabH B⁺ | | | | | | | |
| ML103 | ΔfadD, uc_TE⁺, | 0.2 | 12 | 10 | 0.05 | 0.091 | 0.14 | 35.76 |
| (pXZM12, pBHE2) | se_prpE⁺, sa_ fabH⁺ | | | | | | | |
| ML103 | ΔfadD, uc_TE⁺, | 0.2 | 12 | 10 | 0.32 | 0.4 | 0.72 | 44.06 |
| (pXZM12, pBHE3) | se_prpE⁺, bs_ fabH A⁺ | | | | | | | |
| ML103 | ΔfadD, uc_TE⁺, | 0.2 | 12 | 10 | 0.46 | 1.85 | 2.31 | 19.75 |
| (pXZM12, pBHE4) | se_prpE⁺, sp_dpsC⁺ | | | | | | | |
| ML103 | ΔfadD, rc_TE⁺, | 0.5 | 12 | 10 | 0.57 | 2.17 | 2.74 | 20.74 |
| (pXZ18, pBHE1) | se_prpE⁺, bs_ fabH B⁺ | | | | | | | |
| ML103 | ΔfadD, rc_TE⁺, | 0.5 | 12 | 10 | 0.072 | 0.21 | 0.28 | 25.3 |
| (pXZ18, pBHE2) | se_prpE⁺, sa_ fabH⁺ | | | | | | | |
| ML103 | ΔfadD, rc_TE⁺, | 0.5 | 12 | 10 | 0.39 | 0.35 | 0.73 | 52.93 |
| (pXZ18, pBHE3) | se_prpE⁺, bs_ fabH A⁺ | | | | | | | |
| ML103 | ΔfadD, rc_TE⁺, | 0.5 | 12 | 10 | 0.16 | 2.34 | 2.5 | 6.39 |
| (pXZ18, pBHE4) | se_prpE⁺, sp_dpsC⁺ | | | | | | | |
| ML103ΔfabH | ΔfadD, ΔfabH, | 0.2 | 12 | 10 | 0.56 | 0.68 | 1.23 | 45.2 |
| (pXZM12, pBHE1) | uc_TE⁺, se_prpE⁺, | | | | | | | |
| | bs_ fabH B⁺ | | | | | | | |
| ML103ΔfabH | ΔfadD, ΔfabH, | 0.2 | 12 | 10 | 1 | 0.36 | 1.36 | 73.45 |
| (pXZM12, pBHE2) | uc_TE⁺, se_prpE⁺, | | | | | | | |
| | sa_ fabH⁺ | | | | | | | |
| ML103ΔfabH | ΔfadD, ΔfabH, | 0.2 | 12 | 10 | 0.18 | 0.049 | 0.23 | 78.59 |
| (pXZM12, pBHE3) | uc_TE⁺, se_prpE⁺, | | | | | | | |
| | bs_ fabH A⁺ | | | | | | | |
| ML103ΔfabH | ΔfadD, ΔfabH, | 0.2 | 12 | 10 | 0.027 | 0.099 | 0.13 | 16.75 |
| (pXZM12, pBHE4) | uc_TE⁺, se_prpE⁺, | | | | | | | |
| | sp_dpsC⁺ | | | | | | | |
| ML103ΔfabH | ΔfadD, ΔfabH, | 0.5 | 12 | 10 | 0.81 | 1.47 | 2.28 | 35.64 |
| (pXZ18, pBHE1) | rc_TE⁺, se_prpE⁺, | | | | | | | |
| | bs_ fabH B⁺ | | | | | | | |
| ML103ΔfabH | ΔfadD, rc_TE⁺, | 0.5 | 12 | 10 | 1.2 | 0.79 | 1.99 | 60.27 |
| (pXZ18, pBHE2) | se_prpE⁺, sa_ fabH⁺ | | | | | | | |
| ML103ΔfabH | ΔfadD, ΔfabH, | 0.5 | 12 | 10 | 0.61 | 0.12 | 0.73 | 83.17 |
| (pXZ18, pBHE3) | rc_TE⁺, se_prpE⁺, | | | | | | | |
| | bs_ fabH A⁺ | | | | | | | |
| ML103ΔfabH | ΔfadD, ΔfabH, | 0.5 | 12 | 10 | 0.004 | 0.065 | 0.069 | 5.95 |
| (pXZ18, pBHE4) | rc_TE⁺, se_prpE⁺, | | | | | | | |
| | sp_dpsC⁺ | | | | | | | |
| ML191ΔfabH | ΔpfkA, ΔfadD, | 0.1 | 12 | 10 | 0.84 | 0.13 | 0.97 | 86.6 |
| (pXZmch, pBHE2) | ΔfabH, ch _TE⁺, | 0.5 | 12 | 10 | 0.75 | 0.059 | 0.8 | 93.75 |
| | se_prpE⁺, sa_ fabH⁺ | 0.5 | 12 | 20 | 0.59 | 0.090 | 0.68 | 86.76 |

uc_TE⁺: overexpression of acyl-ACP thioesterase from U. californica;
rc_TE⁺: overexpression of acyl-ACP thioesterase from R. communis;
ch_TE+: overexpression of acyl-ACP thioesterase from C. hookeriana;
se_prpE⁺: overexpression of propionyl-CoA synthetase from S. enterica;
bs_fabH B⁺: overexpression of β-ketoacyl-ACP synthase III B from B. subtilis;
sa_ fabH⁺: overexpression of β-ketoacyl-ACP synthase III B from S. aureus;
bs_ fabH A⁺: overexpression of β-ketoacyl-ACP synthase III A from B. subtilis;
sp_dpsC⁺: overexpression of β-ketoacyl:acyl carrier protein synthase subunit (dpsC) from S. peucetius.

TABLE 18
Total glucose consumption and acetate accumulation at 48 h

| | | Culture Condition | | | Glucose | Acetate | |
|---|---|---|---|---|---|---|---|
| | Relevant | IPTG | Propionate | Arabinose | consumption | accumulation | |
| Strain | genotype | (mM) | (mM) | (mM) | (mM) | (mM) | OD₆₀₀ |
| ML103 | ΔfadD, uc_TE⁺, | 0.2 | 12 | 10 | 80.79 | 5.11 | 6.63 |
| (pXZM12, pBHE1) | se_prpE⁺, bs_ fabH B⁺ | | | | | | |
| ML103 | ΔfadD, uc_TE⁺, | 0.2 | 12 | 10 | 28.1 | 43.03 | 4.19 |
| (pXZM12, pBHE2) | se_prpE⁺, sa_ fabH⁺ | | | | | | |
| ML103 | ΔfadD, uc_TE⁺, | 0.2 | 12 | 10 | 33.86 | 38.44 | 5.35 |
| (pXZM12, pBHE3) | se_prpE⁺, bs_ fabH A⁺ | | | | | | |
| ML103 | ΔfadD, uc_TE⁺, | 0.2 | 12 | 10 | 79.2 | 5.17 | 5.53 |
| (pXZM12, pBHE4) | se_prpE⁺, sp_dpsC⁺ | | | | | | |
| ML103 | ΔfadD, rc_TE⁺, | 0.5 | 12 | 10 | 80.84 | 18.89 | 7.18 |
| (pXZ18, pBHE1) | se_prpE⁺, bs_ fabH B⁺ | | | | | | |
| ML103 | ΔfadD, rc_TE⁺, | 0.5 | 12 | 10 | 33.5 | 57.88 | 4.81 |
| (pXZ18, pBHE2) | se_prpE⁺, sa_ fabH⁺ | | | | | | |
| ML103 | ΔfadD, rc_TE⁺, | 0.5 | 12 | 10 | 46.01 | 46.9 | 7.8 |
| (pXZ18, pBHE3) | se_prpE⁺, bs_ fabH A⁺ | | | | | | |
| ML103 | ΔfadD, rc_TE⁺, | 0.5 | 12 | 10 | 79.33 | 12.56 | 7.05 |
| (pXZ18, pBHE4) | se_prpE⁺, sp_dpsC⁺ | | | | | | |
| ML103ΔfabH | ΔfadD, ΔfabH, | 0.2 | 12 | 10 | 64.67 | 23.49 | 5.73 |
| (pXZM12, pBHE1) | uc_TE⁺, se_prpE⁺, | | | | | | |
| | bs_ fabH B⁺ | | | | | | |
| ML103ΔfabH | ΔfadD, ΔfabH, | 0.2 | 12 | 10 | 64.85 | 31.5 | 5.95 |
| (pXZM12, pBHE2) | uc_TE⁺, se_prpE⁺, | | | | | | |
| | sa_ fabH⁺ | | | | | | |
| ML103ΔfabH | ΔfadD, ΔfabH, | 0.2 | 12 | 10 | 32.45 | 28.64 | 3.74 |
| (pXZM12, pBHE3) | uc_TE⁺, se_prpE⁺, | | | | | | |
| | bs_fabH A⁺ | | | | | | |
| ML103ΔfabH | ΔfadD, ΔfabH, | 0.2 | 12 | 10 | 22.45 | 45.7 | 2.82 |
| (pXZM12, pBHE4) | uc_TE⁺, se_prpE⁺, | | | | | | |
| | sp_dpsC⁺ | | | | | | |
| ML103ΔfabH | ΔfadD, ΔfabH, | 0.5 | 12 | 10 | 79.23 | 19.01 | 5.96 |
| (pXZ18, pBHE1) | rc_TE⁺, se_prpE⁺, | | | | | | |
| | bs_fabH B⁺ | | | | | | |
| ML103ΔfabH | ΔfadD, rc_TE⁺, | 0.5 | 12 | 10 | 79.37 | 22.62 | 8.63 |
| (pXZ18, pBHE2) | se_prpE⁺, sa_ fabH⁺ | | | | | | |
| ML103ΔfabH | ΔfadD, ΔfabH, | 0.5 | 12 | 10 | 46.93 | 45.02 | 6.81 |
| (pXZ18, pBHE3) | rc_TE⁺, se_prpE⁺, | | | | | | |
| | bs_ fabH A⁺ | | | | | | |
| ML103ΔfabH | ΔfadD, ΔfabH, | 0.5 | 12 | 10 | 10.92 | 26.66 | 1.29 |
| (pXZ18, pBHE4) | rc_TE⁺, se_prpE⁺, | | | | | | |
| | sp_dpsC⁺ | | | | | | |
| ML191ΔfabH (pXZ | ΔpfkA, ΔfadD, | 0.1 | 12 | 10 | 76.32 | 6.24 | 6.58 |
| mch, pBHE2) | ΔfabH, ch_TE⁺, | 0.5 | 12 | 10 | 60.33 | 5.34 | 6.93 |
| | se_prpE⁺, sa_ fabH⁺ | 0.5 | 12 | 20 | 55.01 | 10.28 | 7.18 |

uc_TE⁺: overexpression of acyl-ACP thioesterase from U. californica;
rc_TE⁺: overexpression of acyl-ACP thioesterase from R. communis;
ch_TE+: overexpression of acyl-ACP thioesterase from C. hookeriana;
se_prpE⁺: overexpression of propionyl-CoA synthetase from S. enterica;
bs_fabH B⁺: overexpression of β-ketoacyl-ACP synthase III B from B. subtilis;
sa_ fabH⁺: overexpression of β-ketoacyl-ACP synthase III B from S. aureus;
bs_ fabH A⁺: overexpression of β-ketoacyl-ACP synthase III A from B. subtilis;
sp_dpsC⁺: overexpression of β-ketoacyl:acyl carrier protein synthase subunit (dpsC) from S. peucetius.

Each of the following are incorporated by reference in their entireties herein for all purposes.

61/740,959, filed Dec. 21, 2012.

WO2011116279, WO2013059218, US20130084600, US20100009418, US20040199941, US20100009418

Ingram L. O., Chevalier L. S., Gabba E. J., Ley K. D., Winters K., 1977. Propionate-induced synthesis of odd-chain-length fatty acids by Escherichia coli. J. Bacteriol. 131,1023-1025.

Aldor I., Keasling J. D., 2001. Metabolic engineering of poly (3-hydroxybutyrate-co-3-hydroxyvalerate) composition in recombinant Salmonella enterica serovar typhimurium. Biotechnol. Bioeng. 76,08-14.

Wong M. S., Causey T. B., Mantzaris N., Bennett G. N., San K. Y., 2008. Engineering poly (3-hydroxybutyrate-co-3-hydroxyvalerate) copolymer composition in E. coli. Biotechnol. Bioeng. 99,919-928.

Choi K. H., Heath R. J., Rock C. O., 2000. Beta-ketoacyl-acyl carrier protein synthase III (FabH) is a determining factor in branched-chain fatty acid biosynthesis. J Bacteriol. 182(2),365-370.

Smirnova N., Reynolds K. A., 2001. Branched-chain fatty acid biosynthesis in Escherichia coli. J Ind. Microbiol. Biotechnol. 27(4),246-251.

Hu Z. H., Valle F., 2012. Enhanced production of fatty acid derivatives. U.S. Pat. No. 8,110,670.

Zhang X., Li M., Agrawal A., San K. Y., 2011. Efficient free fatty acid production in Escherichia coli using plant acyl-ACP thioesterases. Metab. Eng. 13(6),713-722.

San, K. Y., Li M., Zhang X., NSF CBiRC annual report (2011).

Suwannakham, S., Metabolic Engineering For Enhanced Propionic Acid Fermentation By Propionibacterium Acidipropionici: Dissertation (2005), online at https://etd.ohiolink.edu/ap:0:0:APPLICATION_PROCESS%3DDOWNLOAD_ETD_SUB_DOC_ACCNUM:::F1501_ID:osu1111728310%2Cattachment

1) A genetically modified bacteria, said bacteria having:
a) an overexpressed propionyl-CoA synthase gene; and
b) an overexpressed β-ketoacyl-acyl carrier protein synthase III gene with a greater substrate preference for propionyl-coA than acetyl-coA;
wherein said bacteria makes more odd-chain fatty acids than a control bacteria lacking a) and b).

2) The bacteria of claim 1, said bacteria having:
a) an overexpressed prpE gene from Salmonella enterica;
b) an overexpressed β-ketoacyl-acyl carrier protein synthase III gene from Bacillus subtilis (fabH1), Bacillus subtilis (fabH2), Staphylococcus aureus (fabH) or Streptomyces peucetius (dpsC); and
c) a knockout of a native β-ketoacyl-acyl carrier protein synthase III gene.

3) The bacteria of claim 2, wherein said overexpressed genes a) and b) are co-expressed from the same expression vector.

4) The bacteria of claim 1, further comprising a knockout of the native fadD.

5) The bacteria of claim 2, further comprising a knockout of the native fadD.

6) The bacteria of claim 1, further comprising an overexpressed acyl-ACP thioesterase

7) The bacteria of claim 2, further comprising an overexpressed acyl-ACP thioesterase from U. californica, R. communis, or C. hookeriana.

8) The bacteria of claim 4, further comprising an overexpressed acyl-ACP thioesterase from U. californica, R. communis, or C. hookeriana.

9) The bacteria of claim 2, further comprising Δack, Δpta or Δack-pta.

10) The bacteria of claim 4, further comprising Δack, Δpta or Δack-pta.

11) The bacteria of claim 8, further comprising Δack, Δpta or Δack-pta.

12) The bacteria of claim 1, said bacteria selected from E. coli comprising the following genotypes:
ΔfadD, uc_TE⁺, se_prpE⁺, bs_fabH B⁺
ΔfadD, uc_TE⁺, se_prpE⁺, sa_fabH⁻
ΔfadD, uc_TE⁺, se_prpE⁺, bs_fabH A⁺
ΔfadD, rc_TE⁺, se_prpE⁺, bs_fabH A⁺
ΔfadD, ΔfabH, uc_TE⁺, se_prpE⁺, bs_fabH B⁺
ΔfadD, ΔfabH, uc_TE⁺, se_prpE⁺, sa_fabH⁺
ΔfadD, ΔfabH, uc_TE⁺, se_prpE⁺, bs_fabH A⁺
ΔfadD, ΔfabH, rc_TE⁺, se_prpE⁺, bs_fabH B⁺
ΔfadD, rc_TE⁺, se_prpE⁺, sa_fabH⁺
ΔfadD, ΔfabH, rc_TE⁺, se_prpE⁺, bs_fabH A⁺
ΔpfkA, ΔfadD, ΔfabH, ch_TE⁺, se_prpE⁺, sa_fabH⁺
wherein:
uc_TE⁺=overexpression of acyl-ACP thioesterase (TE⁺) from Umbellularia califonica;
rc_TE⁺=TE⁺ from Ricinus communis;
ch_TE+=TE⁺ from Cuphea hookeriana;
se_prpE⁻=overexpression of propionyl-CoA synthetase from Salmonella. enterica;
bs_fabH B⁺=overexpression of β-ketoacyl-ACP synthase III B from Bacillus subtilis;
sa_fabH⁺=overexpression of β-ketoacyl-ACP synthase III B from Staphylococcus aureus;
bs_fabH A⁺=overexpression of β-ketoacyl-ACP synthase III A from Bacillus subtilis; and
sp_dpsC⁺=overexpression of β-ketoacyl:acyl carrier protein synthase subunit from Streptomyces peucetius.

13) The bacteria of claim 1, having the genotype ΔpfkA, ΔfadD, ΔfabH, ch_TE⁺, se_prpE⁺, sa_fabH⁺.

14) The bacteria of claim 1, having the genotype ΔfadD, ΔfabH, uc_TE⁺, se_prpE⁺, sa_fabH⁺.

15) The bacteria of claim 1, having the genotype ΔfadD, ΔfabH, uc_TE⁺, se_prpE⁺, bs_fabH A⁺.

16) The bacteria of claim 1, having the genotype ΔfadD, ΔfabH, rc_TE⁺, se_prpE⁺, bs_fabH A⁺.

17) A method of producing odd-chain fatty acids, comprising culturing a bacteria of claim 1 in a nutrient broth for a period of time sufficient for said bacteria to produce odd-chain fatty acids, and isolating said off chain fatty acids from said nutrient broth or said bacteria, or both.

18) The method of claim 17, wherein propionate is provided in said nutrient broth.

19) The method of claim 17, wherein propionate is provided by overexpressing the genes from Propionibacterium encoding for oxaloacetate transcarboxylase and succinyl CoA transferase, thus allowing the production of propionic acid from pyruvate.
